SEQUENCE LISTING (4 sequences)

SEQ ID NO: 1 - DNA, 23 bp, Homo sapiens
ggtgcggtggttcctcagcctgg

SEQ ID NO: 2 - DNA, 20 bp, Homo sapiens
gggccttgagatacccagtg

SEQ ID NO: 3 - DNA, 26 bp, Homo sapiens
gcacccccaaataaacacacaccgta

SEQ ID NO: 4 - DNA, 26 bp, Homo sapiens
tacggtgtgtgtttatttgggggtgc

(19) United States
(12) Patent Application Publication
(10) Pub. No.: US 2013/0184199 A1
(43) Pub. Date: Jul. 18, 2013

(54) INSULIN RECEPTOR INDUCED ELASTIN PRODUCTION

(71) Applicants: HUMAN MATRIX SCIENCES, LLC, Visalia, CA (US); THE HOSPITAL FOR SICK CHILDREN, Toronto (CA)

(72) Inventors: Thomas F. MITTS, Visalia, CA (US); Aleksander HINEK, Toronto, (CA)

(73) Assignees: THE HOSPITAL FOR SICK CHILDREN, Toronto (CA); HUMAN MATRIX SCIENCES, LLC, Visalia, CA (US)

(21) Appl. No.: 13/735,824

(22) Filed: Jan. 7, 2013

Related U.S. Application Data
(60) Provisional application No. 61/584,105, filed on Jan. 6, 2012.

(51) Int. Cl. A61K 38/28

(52) U.S. Cl. CPC A61K 38/28
USPC 514/1.9; 514/5.9; 514/5.3; 435/375

(57) ABSTRACT

Compositions and methods for modulating the deposition of elastin by administering compositions including insulin receptor agonists are described herein.

This application claims the benefit of U.S. Provisional Application No. 61/584,105 filed Jan. 6, 2012, incorporated herein by reference in its entirety.

### SUMMARY

Disclosed are compositions and methods for regulating the deposition of elastin in cells, tissues, and organs. Such compositions include insulin receptor agonists such as insulin that are provided to a patient in need thereof to regulate the deposition of elastin in cells, tissues, and organs. In certain embodiments insulin receptor agonists, including insulin, are administered systemically or locally to stimulate the production of elastin. In certain examples, cells, tissues and organs, are exposed to a concentration of 0.5-10 nM insulin, insulin analogue, insulin agonist, or insulin fragment.

In yet other embodiments, the therapeutic concentration of insulin or insulin receptor agonists does not up-regulate deposition of collagen type I and fibronectin or stimulate cellular proliferation. In certain other examples, the therapeutic level of insulin or insulin agonist does not induce cross-reactivity with the Insulin-like Growth Factor-1 Receptor (IGF-1R). In yet other embodiments, the therapeutic levels of insulin or insulin agonist up-regulate elastin gene transcription and the enhancement of tropoelastin secretion. In yet other embodiments, therapeutic levels of insulin or insulin agonists promote association of tropoelastin with its 67-kDa EBP/S-Gal chaperone thereby facilitating secretion.

When the disclosed methods and compositions are used in humans and animals, pathologic dysregulation of elastin production can be treated. For example, therapeutic applications include treatment of atherosclerosis, ischemic heart disease, peripheral vascular disease, cerebrovascular disease, ulceration, chronic wound care, ischemic tissue repair, metabolic syndrome, diabetic-associated retinopathy, diabetic-associated radiculopathy, and diabetic-associated neuropathy. In certain other embodiments, the disclosed compositions and treatments can be used to treat primary elastinopathies such as supravalvular aortic stenosis (SVAS), Williams-Beuren syndrome (WBS), Cutis Laxa, and secondary elastinopathies such as Marfan disease, GM-1-gangliosidosis, Morquio B, Hurler disease, Costello syndrome, Ehlers Danlos syndrome, and pseudoxanthoma elasticum (PXE). In yet other embodiments, the methods and compositions disclosed can be used to treat wrinkles, reduce scarring, and promote healing of skin.

### DESCRIPTION OF DRAWINGS

The file of this patent contains at least one photograph or drawing executed in color. Copies of this patent with color drawing(s) or photograph(s) will be provided by the Patent and Trademark Office upon request and payment of the necessary fee.

For a fuller understanding of the nature and advantages of the present invention, reference should be made to the following detailed description taken in connection with the accompanying drawings, in which:

FIG. 1. Low concentrations of insulin induce production of elastic fibers in cultures of cells derived from human aorta. A: Representative micrographs depict immunodetected elastic fibers (green) in 24-hour cultures (second passage) of SMCs derived from human aortic media. Morphometric evaluation (B) and quantitative assay of the metabolically labeled insoluble elastin (C) demonstrate that low concentrations (0.5 to 100 nmol/L) of insulin induce new elastogenesis in a dose-dependent manner. Nuclei were counterstained with propidium iodide (red). *P<0.01. **P<0.001. D: Representative micrographs demonstrate that the second passage of cells derived from human aortic media, exhibiting SMC-specific-actin, and the parallel cultures derived from aortic adventitia, containing primarily fibroblasts and only single-actin-positive SMCs, deposit multiple immunodetected elastic fibers in response to 48-hour treatment with 10 nmol/L insulin. Nuclei were counterstained with DAPI (blue). E: Quantitative assay measuring incorporation of [3H]-thymidine and immunodetection of Ki-67 proliferative antigen in 48-hour cultures of AoSMCs (F, G) demonstrate that treatment with 0.5 to 100 nmol/L insulin did not induce any up-regulation of their basic proliferative rate. Nuclei were counterstained with hematoxylin (blue). All cells were maintained in medium with 2% FBS. Scale bars 15 μm. All results are derived from three separate experiments in which quadruplicate cultures from each experimental group were assessed.

FIG. 2. Insulin stimulates elastogenesis through exclusive activation of the insulin receptors and the downstream signaling pathway that includes PI3K. A: Results of immunoprecipitations with antibodies recognizing insulin receptor (anti-IR beta subunit) and IGF-IR (anti-IGF-1 beta subunit) followed by immunoblotting with anti-phospho-Tyr antibody demonstrate that 30-minute treatment of cultured AoSMCs (second passage, maintained in serum-free medium) with 0.5 to 10 nmol/L insulin induced dose-dependent phosphorylation of the insulin receptor but did not affect phosphorylation of the IGF receptor, which phosphorylation could only be induced using 10 nmol/L IGF-1. B: Results of RT-PCR assays reveal that 8-hour treatment of AoSMCs (maintained in serum-free medium) with 10 nmol/L insulin induced significant up-regulation in levels of tropoelastin mRNA. This effect of 10 nmol/L insulin was not observed in cultures pretreated for 30 minutes and subsequently maintained in the presence of insulin receptor kinase inhibitor (Ag1024) or PI3K inhibitor (LY294002). In contrast, pretreatment and consecutive incubation of parallel cultures using the specific inhibitor of IGF-1R tyrosine kinase (picropodophyllin) did not inhibit the elastogenic effects induced by 10 nmol/L insulin. Representative photomicrographs of 24-hour cultures of human AoSMCs (maintained in medium with 2% FBS) immunostained with anti-tropoelastin antibody (green) (scale bars 15 μm) (C) and results of their morphometric evaluation (D), as well as assessment of [3H]-valine-labeled insoluble elastin (E) confirmed that inhibition of insulin receptor kinase and PI3K (left) but not IGF-1R kinase (right) eliminated the elastogenic effects of 10 nmol/L insulin. The elastogenic effects of 10 nmol/L IGF-1 observed in parallel cultures were also eliminated after inhibition of IGF-1R kinase or PI3K. All results are derived from three separate experiments in which quadruplicate cultures from each experimental group were assessed. *P<0.001.

FIG. 3. Final steps in the insulin-induced elastogenic signaling pathway differ from those induced by IGF-1 and involve dissociation of FoxO1 transcription inhibitor from the elastin gene promoter. A: Representative photomicrographs depict immunodetected elastic fibers (green) in 24-hour cultures of the third passage of human AoSMCs (maintained in the presence of 2% FBS) and demonstrate that the specific inhibition of cdk-2 with CVT313 abolished the elastogenic effect in cultures treated with 10 nmol/L IGF-1 but did not diminish the elastogenic effect of 10 nmol/L insulin (B). Nuclei were counterstained with propidium iodide (red). Scale bars 15 μm. *P<0.001. C: Western blotting using indicated antibodies demonstrate that in contrast to 30-minute treatment with 10 nmol/L IGF-1, parallel treatment with 10 nmol/L insulin did not induce phosphorylation of cdk-2 on tyrosine 160 or promote site-specific phosphorylation of retinoblastoma protein on threonine 821. D: The CAAATAA sequence localized at position 1948 in the elastin gene promoter is highly homologous to the FRE detected in other insulin-modulated genes. E: Dynabead-immobilized synthetic CAAATAA DNA probe (reflecting FRE detected in elastin gene promoter) sequestered FoxO1 protein from the nuclear extracts of AoSMCs cultured in the absence of insulin. This DNA probe could not sequester FoxO1 from nuclear extracts of cells treated for 30 minutes with 10 nmol/L insulin. In contrast, this probe bound abundant FoxO1 protein from the nuclear extracts of cells preincubated using the PI3K inhibitor LY294002, and consecutive treatment with insulin could not reverse this interaction. All results are derived from three separate experiments in which quadruplicate cultures from each experimental group were assessed.

FIG. 4. Independent of its genomic elastogenic effect, insulin enhances tropoelastin secretion and facilitates association between tropoelastin and its S-Gal/EBP chaperone. Results of two quantitative assays measuring the levels of immunoprecipitated tropoelastin in confluent cultures of AoSMCs that were first maintained for 2 hours in serum-free medium containing [3H]-valine and the lysyl oxidase inhibitor (BAPN) and then incubated for 1 hour in the presence or absence of 10 nmol/L insulin. Assays demonstrated that extracts of AoSMCs incubated in the presence of 10 nmol/L insulin contain significantly less metabolically labeled soluble tropoelastin (immunoprecipitated (A) or Western blotted (B) using anti-tropoelastin antibody) than do their untreated counterparts. Conversely, their conditioned media contain significantly more immunodetected soluble tropoelastin when compared with untreated cultures. These insulin-induced changes in levels of newly synthesized tropoelastin could not be observed in parallel cultures, in which activity of PI3K was inhibited by 1 hour preincubation with LY294002. C: Results of experiments in which confluent cultures of AoSMCs (second passage) were incubated in serum-free medium for only 20 minutes in the presence or absence of 10 nmol/L insulin demonstrate that the cell extract of insulin-treated cultures do not demonstrate any increase in basic levels of intracellular S-Gal/EBP or tropoelastin detected using Western blot analysis. D: At the same time, the insulin-treated cells contain significantly more S-Gal/EBP, coimmunoprecipitated using anti-tropoelastin antibody, when compared with their untreated counterparts. In contrast, insulin could not induce the same effect in parallel cultures pretreated for 30 minutes using PI3K inhibitor. All results were derived from three separate experiments in which quadruplicate cultures from each experimental group were assessed. *P<0.001. E: Representative micrographs depict cultures of AoSMCs (third passage) subjected to double immunostaining with anti-S-Gal/EBP (red fluorescence) and anti-tropoelastin antibodies (green fluorescence). Results show that in untreated cells, most tropoelastin (green) is localized to the central endoplasmic reticulum and is separate from peripheral endosomes (red) containing S-Gal/EBP. In contrast, cells treated for 20 minutes with 10 nmol/L insulin reveal co-localization of tropoelastin and its S-Gal/EBP chaperone in the peripheral secretory vesicles (yellow fluorescence). Nuclei were counterstained with DAPI (blue). Scale bars 5 μm.

### DESCRIPTION

Elastic fibers are major components of Extra Cellular Matrix “ECM”, providing tissues with resilience and elastic recoil. They are composed of a microfibrillar scaffold made up of several glycoproteins and a core consisting of the unique polymeric protein, elastin. Elastin is formed extracellulary after the lysyl oxidase-dependent cross-linking of lysine residues present in the multiple molecules of the 72-kDa precursor, tropoelastin, secreted from fibroblasts or smooth muscle cells. The newly synthesized tropoelastin has to be escorted through the secretory pathways by the 67 kDa elastin binding protein (EBP), identified as the catalytically inactive spliced variant of β-galactosidase (S-Gal). This molecular chaperone (EBP/S-Gal) protects hydrophobic and unglycosylated tropoelastin molecules from the premature self-aggregation and proteolytic degradation and assures their orderly extracellular assembly upon the microfibrillar scaffold of elastic fibers.

The elastic fibers present in adult tissues (produced during the late fetal and early postnatal life) constitute the most durable components of the ECM. However, certain metabolic and environmental factors, as well as local injuries or inflammations may induce a progressive loss of elastic fibers in blood vessels, lungs, heart, skin and the framework of other organs. Interestingly, during the compensatory remodeling of the affected tissues the lost elastic fibers are often replaced by the stiff collagen fibers.

Pathologic remodeling of metabolically or mechanically injured blood vessels characterized by the lack of new elastogenesis, is associated with the development of atherosclerotic plaques and vascular occlusions leading to cardiac infarctions and cardiomyopathy, as well contributing to delayed wound healing, skin atrophy and necrosis. All these pathologies are particularly frequent and develop faster in diabetic patients. The development of cardiovascular complications of diabetes were traditionally linked to the metabolic consequences of hyperglycemia vascular endothelial dysfunction, and inflammation. However, it has been observed that there is a peculiar lack of new elastogenesis after coronary artery bypass and after peripheral vascular surgery in diabetic patients.

Diabetes mellitus is associated with progression of atherosclerosis, development of peripheral angiopathies, cardiomyopathy, arterial hypertension and delayed wound healing. However, the mechanistic link between insulin deficiency and impaired elastogenesis in the diabetic tissues has remained unknown. The impaired initial elastogenesis and rapidly progressing loss of existing elastic fibers constitutes the common denomination in mechanisms leading to development of both, arterial hypertension and atherosclerotic lesions in diabetic patients. Consequently, insulin has not been recognized as a factor that would regulate primary elastogenesis in skin, arteries or myocardium.

As such, there has never been any treatment option available to address the pathological elastogenic responses in patients via the insulin receptor. Similarly, targeted therapies for modulating pathological elastogenic responses via the insulin receptor have not been available.

Disclosed herein are methods and compositions useful for inducing elastogensis. Such methods and compositions comprise activating the insulin receptor thereby increasing or otherwise modulating the production of elastin. As described herein without limitation, activation of elastogenesis by insulin receptor agonists, such as insulin itself or other agonists, can be used to treat and prevent disease in humans and animals.

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must be noted that as used herein, and in the appended claims, the singular forms “a,” “an,” and “the” include plural reference unless the context clearly dictates otherwise. Thus, for example, reference to a “fibroblast” is a reference to one or more fibroblasts and equivalents thereof known to those skilled in the art.

As used herein, all claimed numeric terms are to be read as being preceded by the term, “about,” which means plus or minus 10% of the numerical value of the number with which it is being used. Therefore, a claim to “50%” means “about 50%” and encompasses the range of 45%-55%.

“Administering” when used in conjunction with a therapeutic means to administer a therapeutic directly into or onto a target tissue, or to administer a therapeutic to a patient whereby the therapeutic positively impacts the tissue to which it is targeted. Thus, as used herein, the term “administering,” when used in conjunction with aldosterone or any other composition described herein, can include, but is not limited to, providing aldosterone locally by administering aldosterone into or onto the target tissue, providing aldosterone systemically to a patient by, for example, intravenous injection whereby the therapeutic reaches the target tissue or providing aldosterone in the form of the encoding sequence thereof to the target tissue (e.g., by so-called gene-therapy techniques). “Administering” a composition may be accomplished by any mode including parenteral administration including injection, oral administration, topical administration, or by any other method known in the art including for example electrical deposition (e.g., iontophoresis) and ultrasound (e.g., sonophoresis). In certain embodiments, the compositions described herein may be administered in combination with another form of therapy, including for example radiation therapy, infrared therapy, ultrasound therapy, or any other therapy know in the art or described herein.

In certain embodiments, the compositions may be combined with a carrier. A “carrier” as used herein may include, but is not limited to, an irrigation solution, antiseptic solution, other solution time released composition, elution composition, bandage, dressing, colloid suspension (e.g., a cream, gel, or salve) internal or external dissolvable sutures, dissolvable beads, dissolvable sponges and/or other materials or compositions known now or hereafter to a person of ordinary skill in the art.

The term “animal” as used herein includes, but is not limited to, humans and non-human vertebrates, such as wild, domestic, and farm animals.

The term “improves” is used to convey that the present invention changes either the appearance, form, characteristics and/or the physical attributes of the tissue to which it is being provided, applied or administered. The change in form may be demonstrated by any of the following, alone or in combination: enhanced appearance of the skin, increased softness of the skin, increased turgor of the skin, increased texture of the skin, increased elasticity of the skin, decreased wrinkle formation and increased endogenous elastin production in the skin, increased firmness and resiliency of the skin.

The term “inhibiting” includes the administration of a compound of the present invention to prevent the onset of the symptoms, alleviating the symptoms, or eliminating the disease, condition or disorder.

By “pharmaceutically acceptable,” it is meant that the carrier, diluent or excipient must be compatible with the other ingredients of the formulation and not deleterious to the recipient thereof. By “excipient,” it is meant any inert or otherwise non-active ingredient, which can be added to the active ingredient which may improve the overall composition's properties, such as improving shelf-life, improving retention time at the application site, improving flowability, improving consumer acceptance, et alia.

Unless otherwise indicated, the term “skin” means that outer integument or covering of the body, consisting of the dermis and epidermis and resting upon subcutaneous tissue.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to increase production of elastin or the deposition of elastic fibers. For example, a therapeutic effect may be demonstrated by increased elastogensis, increased cellular proliferation, increased digestion or resorption of scar material, reduction of symptoms and sequellae as well as any other therapeutic effect known in the art. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, the physical characteristics of the patient (height, weight, etc.), and the condition being treated. It will be understood that the effective amount administered will be determined by the physician in light of the relevant circumstances, including the condition to be treated, the choice of compound to be administered, and the chosen route of administration, and therefore, the dosage ranges provided are not intended to limit the scope of the invention in any way. A “therapeutically effective amount” of compound of this invention is typically an amount such that when it is administered in a physiologically tolerable excipient composition, it is sufficient to achieve an effective systemic concentration or local concentration in the tissue.

In certain embodiments, the local cellular concentration of insulin or insulin other insulin receptor agonist is in the range of 0.5-10 nM/L. Those of skill in the art recognize that such a concentration is easily convertible among equivalents. For example, where the molecular weight of human insulin is 5808 MW, the solute mass in a 1 nM/L solution is 5,808 ng/L. Similarly, the use of the volume in the denominator is not necessary to describe the molarity of a solution. Therefore, as in the above example, a 1 nM solution of insulin would comprise insulin at a ratio of 5,808 ng/L of water.

Those of skill in the art recognize that the agonistic function of any ligand can also be described in terms of the binding kinetics of that ligand to its cognate receptor. With regard to the insulin receptor (CD220), it is a transmembrane receptor that is activated by insulin, IGF-I, IGF-II and belongs to the large class of tyrosine kinase receptors. Biochemically, the insulin receptor is encoded by a single gene INSR, from which alternate splicing during transcription results in either IR-A or IR-B isoforms. Downstream post-translational events of either isoform result in the formation of a proteolytically cleaved α and β subunit, which upon combination are ultimately capable of homo or hetero-dimerization to produce the ≈320 kDa disulfide-linked transmembrane insulin receptor. The binding relationship between the insulin receptor “IR” and ligand shows allosteric properties. According to models, each IR may bind to an insulin molecule (which has two binding surfaces) via 4 locations, being site 1, 2, (3/1′) or (4/2′). As each site 1 proximally faces site 2, upon insulin binding to a specific site, ‘crosslinking’ via ligand between monomers is predicted to occur (i.e. as [monomer 1 Site 1-Insulin-monomer 2 Site (4/2′)] or as [monomer 1 Site 2-Insulin-monomer 2 site (3/1′)]).

As such, the concentrations of a ligand such as insulin necessary to agonize the insulin receptor and thereby produce elastogenesis can be 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10.0 nM, 20 nM, 30 nM, 40 nM, 50 nM, 60 nM, 70 nM, 80 nM, 90 nM, 100 nM, or higher. Those of skill in the art recognize that distribution of insulin in the body and throughout the tissues is not uniform, so that an in situ concentration of 0.5 nM-100 nM inducing elastogenesis by means of the IR may be independent of the systemic concentration or dose of an IR agonist. As such, it is contemplated that systemic administration of insulin or other IR agonist, for example, can be adjusted to target individual classes of cells, individual tissues, and individual organs depending on the type of disease and symptoms of that disease. It is also contemplated that insulin can be delivered locally to a site such as a wound vessel in need of elastogenesis so that the concentration in situ is 0.5 nM-10 nM.

In certain embodiments, the insulin receptor agonist may interact with cells expressing the insulin receptor in a ligand-specific manner so as not to significantly induce cross-reactivity with any other receptor having insulin-binding capability, such as the Insulin-Like Growth Factor-1 Receptor (IGFR-1R) which has 1000× lower affinity for insulin compared to the insulin receptor. In certain embodiments, the dosage of insulin stimulates elastogenesis without the undesirable stimulation of the IGFR-1R which can cause collagen type I and fibronectin production or can cause cellular proliferation. In certain embodiments, therapeutically active concentrations of insulin required to activate insulin receptor elastogenesis are lower than those required to cross react with the IGF-1R. In certain embodiments, the dosage window balancing such effects is termed “low dose” insulin treatment and comprises a dosage creating a concentration of insulin of 0.5-10 nM locally. Such local concentrations can be achieved by any means known in the art including deposition injection, topical administration, perfusion and others. As such, it is also contemplated in the disclosure that when insulin is administered to induce elastogenesis, the dosages are adjusted so as to avoid stimulation of the IGF-1R and any concomitant effects opposing the elastogenic action of the insulin, such as for example, avoiding production of collagen type I and fibronectin or stimulating cellular proliferation.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As disclosed herein, the treatment of an elastinopathy can be achieved by delivering a therapeutic dosage to agonize the insulin receptor, but not so as to agonize other receptors capable of binding insulin, such as IGF-1R. Consequently, the concentration of insulin to be delivered to a patient may be significantly less than the concentration necessary to regulate blood sugar levels, termed herein “non-glycemic doses” for such lowered insulin doses. Thus, in certain embodiments, the concentration of insulin including elastogensis would be considered “low dose” administration relative to the indicated uses of insulin normally used to regulate blood glucose levels. As such, a patient can have normal blood glucose, elevated blood glucose or diminished blood glucose but still derive therapeutic effects from non-glycemic doses of insulin that are independent of insulin's effects on blood sugar regulation. Thus, it is contemplated that non-diabetic patients may also benefit from administration of insulin receptor agonists such as insulin.

To the extent that a patient has either diminished blood insulin levels as in Type 1 diabetes (hypoglycemia) or insulin resistance as in Type 2 diabetes (hyperglycemia), the administration insulin to achieve elastogenesis independent of the glycemic effects would also be within the scope of the disclosure. For example, in a Type 1 diabetic patient, the dosage of insulin required to induce elastogenesis would be below the physiologic dosage of insulin necessary to induce a glycemic response. It is thus contemplated that the disclosed methods and compositions can successfully treat diseases involving elastinopathies. For example, insulin therapy of atherosclerotic lesions in patients with type 1 diabetes is especially useful, because induction of new elastic fibers would mechanically stabilize the developing plaques and prevent the imminent arterial occlusions. Those of skill in the art recognize that the intended effects of administering a therapeutic treatment should avoid non-specific or undesirable side-effects. As such, the local administration of insulin would be at a concentration of 0.5 nM-10 nM when administering “low dose” insulin therapy.

Generally speaking, the term “tissue” refers to any aggregation of similarly specialized cells which are united in the performance of a particular function. As used herein, “tissue,” unless otherwise indicated, refers to tissue which includes elastin as part of its necessary structure and/or function. For example, connective tissue which is made up of, among other things, collagen fibrils and elastin fibrils satisfies the definition of “tissue” as used herein. Additionally, elastin appears to be involved in the proper function of blood vessels, veins, and arteries in their inherent visco-elasticity.

The composition of various embodiments may include any form of insulin, insulin analogue, fragment, alpha or beta chain, pro-insulin, pre-pro-insulin, isoform, species variant, or other insulin receptor agonist known in the art, including, for example, insulin itself. Without being bound by theory, the role of insulin in elastogenesis is disclosed herein to be modulated and induced by the insulin receptor. As such, any molecule targeted to the IR is encompassed by this disclosure. Other embodiments include pharmaceutical compositions, including an insulin receptor agonist and a pharmaceutically acceptable carrier, diluent, or excipient, and in certain embodiments, the compositions or pharmaceutical compositions may include secondary active agents which enhance or improve the function of the insulin receptor agonist. Such compositions may be formulated in any way. For example, in various embodiments, the compositions may be formulated as a liquid, solid, gel, lotion or cream, and the formulation of the composition may vary among embodiments depending on the mode of administration of the compositions.

In various embodiments, the insulin receptor agonist may interact with cells, such as, for example, smooth muscle cells, and the like, and induce the production of elastin by these cells or increase the deposition of the elastin into the extracellular space surrounding these cells.

Without wishing to be bound by theory, enhancing the ability of a cell to produce elastic when stimulated by an insulin receptor agonist may increase the net deposition of elastin fibers in treated tissue thereby enhancing the effectiveness of such treatment. By “increased expression,” it is intended to mean an effect on any pathway that leads to an increase of the number of functional protein molecules, and includes for example, increased IR mRNA synthesis, increased IR mRNA stability, increased anabolism of the protein, decreased catabolism of the protein, and any other pathway by which expression can be increased. By “increased sensitivity,” it is intended to mean increasing the responsiveness of the protein to its ligand, which can occur in any manner including crosslinking of receptors, conformational changes in the receptors, phosphorylation/dephosphorylation of the receptor, or any other mechanism by which sensitivity can be increased.

In yet other examples where endogenous insulin concentrations exceed the requisite concentration necessary to induce elastogenesis via the insulin receptor, glucogon or other oppositional agents to gluconeogensis may be used to modulate the concentration of systemic insulin in order to modulate IR expression and sensitivity. Similarly, additional agents may be administered or adjusted, such as for example changing the patient's blood glucose levels, which can regulates the expression of IR receptor in an individual.

The compositions described in the embodiments above may be administered to any tissue in need of enhanced elastin deposition. For example, in some embodiments, such compositions may be administered to sites of ischemic injury, neuropathy, inflammation, wounds, arteriosclerosis, ulcers, scar, wrinkles and other sites in need of increased elastogenesis. It is contemplated that any cell or tissue expressing insulin receptors be the target for therapy including without limitation blood vessels, the heart, nerves, and skin. In other embodiments, the composition may be administered to cells and tissues associated with the gastrointestinal tract or genitourinary system.

Methods of embodiments generally include administering a composition or pharmaceutical composition including an insulin receptor agonist to a subject or patient in need of treatment. Pharmaceutical compositions useful in various embodiments may be administered to treat, ameliorate, or alleviate symptoms associated with various diseases that may be identified by inability to produce elastin or elastin fibers, or functional elastin or elastin fibers, loss of functional elastin or elastin fibers, or the lack or loss of deposition of elastin or elastin fibers in the subject's tissue. For example, therapeutic applications include treatment elastinopathy, defined as a disease having abnormal production of elastin. Such elastinopathies include, atherosclerosis plaques and subsequent occlusions of coronary arteries, aorta, and peripheral arteries, where the proliferating and migrating smooth muscle cells (SMCs) cannot produce adequate elastin, ischemic neuropathy, ischemic heart disease, emphysema, peripheral vascular disease, cerebrovascular disease, ulceration, chronic wounds, ischemic tissue, metabolic syndrome, diabetic-associated retinopathy, diabetic-associated radiculopathy, and diabetic-associated neuropathy. In certain other embodiments, the disclosed compositions and treatments can be used to treat primary elastinopathies such as supravalvular aortic stenosis (SVAS), Williams-Beuren syndrome (WBS), Cutis Laxa, and secondary elastinopathies such as Marfan disease, GM-1-gangliosidosis, Morquio B, Hurler disease, Costello syndrome, Ehlers Danlos syndrome, and pseudoxanthoma elasticum (PXE). In yet other embodiments, the methods and compositions disclosed can be used to treat wrinkles, reduce scarring, and promote healing of skin. Contemplated treatments also include diseases of the skin, such as, but not limited to, aging, stretch marks, overly stretched skin, sun damaged skin, and scar tissue.

The pharmaceutical composition may be administered by any method known in the art including, for example, systemic administration, local administration, and topical administration. Various embodiments, therefore, include pharmaceutical compositions having an insulin receptor agonist, and a pharmaceutically acceptable carrier, diluent or excipient, or an effective amount of a pharmaceutical composition including an insulin receptor agonist, and a pharmaceutically acceptable carrier, diluent or excipient.

The compounds of the various embodiments may be administered in a conventional manner by any route by which they retain activity. For example, an insulin receptor agonist may be administered by routes including, but not limited to, topical, parenteral, subcutaneous, intravenous, intraperitoneal, transdermal, oral, buccal, inhalation, depot injection, or implantation. Thus, modes of administration for the compounds (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal and topical forms such as patches and creams.

Specific modes of administration will depend on the indication and other factors including the particular compound being administered. The selection of the specific route of administration and the dose regimen is to be adjusted or titrated by the clinician according to methods known to the clinician in order to obtain the optimal clinical response. For example, in embodiments wherein the compositions are used to treat skin, wounds and ulcers for example, the compositions may be administered topically using, for example, a lotion. In other embodiments wherein the compositions are used to treat a disease having systemic effects such as vascular disease, and ischemic heart injury among others, the compositions may be administered systemically, using for example, a tablet or injectable emulsion. In still other embodiments, the compositions may be administered both systemically and topically.

The amount of the compositions of various embodiments to be administered is an amount that is therapeutically effective, and the dosage administered may depend on the characteristics of the subject being treated. For example, the dosage may depend on the particular animal treated, the age, weight, and health of the subject, the types of concurrent treatment, if any, and frequency of treatments. Many of these factors can be easily determined by one of skill in the art (e.g., by the clinician).

Various pharmaceutical formulations include those containing an effective amount of the compounds and a suitable carrier, diluent, or excipient can be in solid dosage forms including, but not limited to, tablets, capsules, cachets, pellets, pills, powders and granules; topical dosage forms including, but not limited to, solutions, powders, fluid emulsions, fluid suspensions, semi-solids, ointments, pastes, creams, lotions, gels, jellies, and foams; and parenteral dosage forms including, but not limited to, solutions, suspensions, emulsions, and dry powders. The active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like.

The means and methods for administration of such pharmaceutical formulations are known in the art and an artisan can refer to various pharmacologic references, such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co., New York (1980) for guidance. For example, in some embodiments, the compounds can be formulated for parenteral administration by injection, and in one embodiment, the compounds can be administered by continuous infusion subcutaneously over a period of about 15 minutes to about 24 hours. In another embodiment, formulations for injection can be presented in unit dosage form, e.g., in ampoules or in multi-dose containers, with an added preservative. In still other embodiments, the compositions can take such forms as suspensions, solutions or emulsions in oily or aqueous vehicles, and can contain formulatory agents such as suspending, stabilizing and/or dispersing agents.

For certain embodiments encompassing oral administration, the compounds can be formulated readily by combining these compounds with pharmaceutically acceptable carriers known in the art. Such carriers enable the compounds to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers. If desired, disintegrating agents, such as, but not limited to, the cross-linked polyvinyl pyrrolidone, agar, or alginic acid or a salt thereof, such as sodium alginate, may be added.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally also include, but are not limited to, push-fit capsules made of gelatin, as well as soft, sealed capsules made of gelatin and a plasticizer, such as glycerol or sorbitol. The push-fit capsules can contain the active ingredients in a mixture with filler such as binders and/or lubricants, such as, for example, talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, for example, tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions, such as, suppositories or retention enemas, for example, containing conventional suppository bases such as cocoa butter or other glycerides.

In addition to the formulations described previously, the compounds of the present invention can also be formulated as a depot preparation. Such long acting formulations can be administered by implantation (for example, subcutaneously or intramuscularly) or by intramuscular injection. Depot injections can be administered at about 1 to about 6 months or longer intervals. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In transdermal administration, the compounds of the present invention can, for example, be applied to a plaster, or can be applied by transdermal, therapeutic systems that are consequently supplied to the organism.

Pharmaceutical compositions of the compounds also can include suitable solid or gel phase carriers or excipients. Examples of such carriers or excipients include but are not limited to calcium carbonate, calcium phosphate, gelatin, and polymers such as, for example, polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

This invention and embodiments illustrating the method and materials used may be further understood by reference to the following non-limiting examples.

### EXAMPLES

In vitro studies described herein, employed cultures of human fibroblasts and aortic smooth muscle cells.

### Materials

Dulbecco's modified Eagle's medium (DMEM), fetal bovine serum (FBS), and other cell culture products were acquired from Gibco-BRL, Life Technologies Corp. (Burlington, ON, Canada). Recombinant human insulin (Humulin R) was purchased from Eli Lilly Canada, Inc. (Toronto, ON, Canada). Recombinant human IGF-1, non-enzymatic cell dissociation solution, proteinase inhibitors, PI3K inhibitor LY294002, glucocorticoid receptor inhibitor RU 486, insulin receptor tyrosine kinase inhibitor AG1024, transforming growth factor-β receptor inhibitor SB431542, lysyl oxidase inhibitor β-aminopropionitrile, transcription inhibitor dichlorobenzimidazole riboside, protein translation inhibitor cycloheximide, anti-phospho-T821-Rb antibody, secondary antibody fluorescein-conjugated goat anti-rabbit and fluorescein-conjugated goat anti-mouse, propidium iodide, and DAPI nuclear stains were purchased from Sigma-Aldrich Corp. (St. Louis, Mo.). Mouse monoclonal antibodies; anti-tropoelastin, anti-α-actin, anti-β-actin, anti-vimentin, anti-pT160-cdk-2, anti-cdk-2, anti-phospho-Tyr (PY-20), and polyclonal antibodies to beta subunits of insulin receptor and IGF-1R were purchased from Santa Cruz Biotechnology, Inc. (Santa Cruz, Calif.). Rabbit polyclonal antibody to tropoelastin was purchased from Elastin Products Co. Inc. (Owensville, Mo.). Polyclonal antibody to collagen type I and monoclonal antibody to fibronectin were purchased from Chemicon International, Inc. (Temecula, Calif.). IGF-IR neutralizing mouse monoclonal antibody and IGF-1R tyrosine kinase inhibitor picropodophyllin, and cdk-2 inhibitor CVT313 were purchased from Calbiochem (San Diego, Calif.). Anti-FoxO1 monoclonal anti-body (C29H4) was purchased from Cell Signaling Technology, Inc. (Beverly, Mass.). Anti-S-GAL/EBP rabbit polyclonal antibody, raised to the elastin/laminin-binding domain of the alternatively spliced variant of β-galactosidase (EBP/S-Gal), was used to detect the 67-kDa elastin-binding protein. Species- and type-specific secondary antibodies conjugated to horseradish peroxidase, an enhanced chemiluminescence kit, the radiolabeled reagent. [³H]-valine and [³H]-thymidine were purchased from Amersham Biosciences Canada, Ltd. (Oakville, ON, Canada). Protein G-bound and M-280 streptavidin-bound Dynabeads were purchased from Invitrogen Canada, Inc. (Burlington, ON, Canada). A DNeasy Tissue system for DNA assay, RNeasy Mini Kit for isolating total RNA, and One-Step RT-PCR Kit were purchased from Qiagen, Inc. (Mississauga, ON, Canada). Nuclear extracts were prepared from cultured cells using the isolation kit from Active Motif, Inc. (Carlsbad, Calif.).

### Culture of Human Aortic SMCs

Human aortic SMCs (AoSMCs) were propagated from small fragments of aortic media collected from autopsy. Guidelines for the protection of human subjects of the US Department of Health and Human Services and of the Declaration of Helsinki were followed in obtaining tissues for this investigation. Cells migrating from the cultured explants of aortic media were routinely passaged (up to three times) via trypsinization using 0.2% trypsin-0.02% EDTA, and were maintained in DMEM supplemented with 5% FBS and 1% antibiotic-antimycotic mix. The second and third passages of cells that migrated from the original explants of aortic media were routinely probed with antibodies recognizing the SMC-specific α-actin to monitor the preservation of the SMC phenotype. All cells were initially plated at 100,000 cells per dish for immediate confluency so they would begin extracellular matrix production. The cell cultures committed to particular experiments were then transferred to DMEM containing either 2% FBS or to serum-free medium before initiation of particular treatments.

### Immunocytochemistry and Morphometry

At the end of treatments, confluent cultures of AoSMCs were fixed in cold 100% methanol at −20° C. for 30 minutes, and blocked with 1% normal goat serum for 1 hour. The cultures were then incubated for 1 hour with 10 μg/mL polyclonal antibodies to tropoelastin and/or with 10 μg/mL monoclonal antibody to SMC-specific α-actin. All cultures were then incubated for another hour with the respective fluorescein-conjugated secondary antibodies (fluorescein-conjugated goat anti-rabbit or anti-mouse). Nuclei were counterstained with red-fluorescent propidium iodide or blue-fluorescent DAPI. The cultures were then mounted in ethanol and examined using a microscope (Eclipse E1000; Nikon Instruments, Inc., Melville, N.Y.) attached to a cooled CCD camera (Retiga EX; Qlmaging Corp., Vancouver, BC, Canada) and a computer-generated morphometric analysis system (Image-Pro Plus software; Media Cybernetics, Inc., Bethesda, Md.). In each analyzed group, 50 low-power fields from three separate cultures were analyzed, and the areas occupied by immunodetectable elastic fibers were expressed as a percentage of the entire analyzed field.

For assessment of intracellular associations between tropoelastin and the 67-kDa elastin binding protein (EBP), cultures of AoSMCs were incubated in serum-free DMEM for 3 hours before treatment with insulin. Cultures fixed for 30 minutes in cold 100% methanol were then blocked with 1% glycine for 15 minutes, followed by 2% bovine serum albumin with 0.1% Triton X-100 for another hour at room temperature. They were finally exposed for 1 hour to the mixture of 5 μg/mL polyclonal anti-S-Gal/EBP and 5 μg/mL monoclonal anti-tropoelastin antibodies. All cultures were then incubated for another hour with the mixture of fluorescein-conjugated and rhodamine-conjugated secondary antibodies. Nuclei were counterstained with DAPI.

### Quantitative Assay of Metabolically Labeled Tropoelastin and Insoluble Elastin

Cells were plated in 35-mm dishes at a density of 100,000 cells per dish and were grown to confluency. Two microcuries [3H]-valine per milliliter fresh medium supplemented with 2% FBS was added 2 hours before the indicated treatments. At the end of each experiment, the conditioned medium was collected and subjected to immunoprecipitation using anti-tropoelastin antibody. The cell layers were washed with PBS and incubated using radioimmunoprecipitation lysis buffer for 10 minutes on ice. After centrifugation, the supernatants were collected and subjected to immunoprecipitation using anti-tropoelastin antibody. The remaining pellets containing extracellular matrix were scraped and boiled in 500 μL 0.1 N NaOH for 30 minutes to solubilize all matrix components except the cross-linked elastin. Final results (counts per minute) reflecting the total amount of [3H]-valine-labeled insoluble elastin in individual cultures were normalized per their DNA contents (assessed using the Qiagen DNeasy Tissue Kit) and expressed as percentage of control values.

### Assessment of Cell Proliferation

Cells were plated in 35-mm culture dishes (100,000 cells per dish) containing DMEM supplemented with 5% FBS. The cultures were grown to 70% to 80% confluency and serum-starved overnight to synchronize the cell cycles. The cultures were then transferred to DMEM containing 2% FBS and 1 μCi [3H]-thymidine/mL, and the quadruplicate cultures were incubated with or without indicated doses of insulin for 48 hours. The total amount of [3H]-thymidine-labeled DNA in individual cultures was then assessed via scintillation counting. The parallel control and insulin-treated cultures were also fixed in cold 100% methanol and ex-posed to antibody detecting the Ki-67 antigen in proliferating cells and then to peroxidase-labeled secondary antibody as previously described. The cultures were counterstained with hematoxylin. In each experimental group, quadruplicate cultures were examined under 200× magnification. The percentage of positively stained cells was determined and averaged over the 30 fields examined.

### Elastin Gene Expression

At the end of the treatments, total RNA was extracted from quadruplicate cultures in each experimental group using the RNeasy Mini Kit. One microgram total RNA from each sample was added to one-step RT-PCR (One-Step RT-PCR Kit; Qiagen, Inc.). The reactions were set up in a total volume of 25 μL. The reverse transcription step was performed for elastin and 18S ribosomal RNA reactions at 50° C. for 30 minutes, followed by 15 minutes at 95° C. The elastin PCR reaction using sense primer 5′GGTGCGGTGGTTCCTCAGCCTGG-3′ (SEQ ID NO 1) and antisense primer 5′GGGCCTTGAGATACCCCAGTG3′ (SEQ ID NO 2). The products were analyzed using ethidium bromide staining and densitometry-based image analysis using an optical system (Gel Doc 1000; Bio-Rad Laboratories, Inc., Hercules, Calif.). The amount of tropoelastin mRNA was standardized relative to the amount of 18S ribosomal RNA and expressed as percentage of control values.

### Western Blotting

At the end of all treatments, cells from quadruplicate cultures in each experimental group were lysed using NP40 cell lysis buffer (Invitrogen Corp.). Thirty micrograms of protein extracts from each sample was suspended in standard sample buffer, resolved on 4% to 12% gradient SDS-PAGE gels, transferred to nitrocellulose membranes, and subjected to Western blotting using antibodies.

### Immunoprecipitation

At the end of indicated treatments, the cultured cells were lysed as described, and 300 μL aliquots of cell extracts containing 300 μL proteins were incubated at 4° C. for 3 hours with the aliquots of protein G bound Dynabeads (Invitrogen Corp.) that were conjugated with either rabbit polyclonal anti-tropoelastin, goat polyclonal anti-Glut10, or rabbit polyclonal anti-S-Gal antibodies. The beads carrying the final immunoprecipitation products were then washed with PBS, resuspended in sample buffer, and boiled for 5 minutes. The released proteins were then resolved on SDS-PAGE gel and subjected to Western blotting using indicated antibodies.

### Determination of Interactions Between FoxO1 and Elastin Gene Promoter

Confluent cultures of human AoSMCs were maintained for 16 hours in DMEM with 2% FBS and then treated in the presence or absence of 10 nmol/L insulin and 10 mmol/L PI3K inhibitor LY294200 for 30-minute periods. Cells were then scraped, and their nuclear extracts were pre-pared using an isolation kit (Active Motif, Inc.). To explore whether the human elastin gene promoter may bind FoxO1 transcription regulating factor and whether such an interaction could be modified by insulin, the DNA oligonucleotides 5′GCACCCCCAAATAAACACACACCGTA-3′ (SEQ ID NO 3) and 5′TACGGTGTGTGTTTATTTGGGGGTGC-3′(SEQ ID NO 4), reflecting a putative Fox-0 binding domain localized in the human elastin gene promoter, were synthesized. The equal molar amounts of both single-stranded DNA oligonucleotides were annealed in Tris-EDTA buffer (10 mmol/L Tris-HCl, 1 mmol/L EDTA (pH 8.0)) to form the double-stranded DNA probe. Then, 20 μL aliquots of M-280 streptavidin containing Dynabeads conjugated with 40 pmol of our biotinylated DNA probe were mixed with samples of nuclear extracts (containing 100 μg protein) derived from cells that were maintained for 30 minutes in the presence or absence of 30 nmol/L insulin and/or 10 μmol/L LY294002. All preparations were subsequently incubated for 2 hours in 200 μL binding buffer (50 mmol/L KCl, 10% glycerol, 20 mmol/L HEPES (pH 7.9), 1 mmol/L MgCl2, 1 mmol/L DTT, 0.1 μg poly(DI-DC) (polydeoxyinosinic-deoxycytidylic), and proteinase inhibitors). After binding, the beads were washed four times in the same binding buffer, without poly(DI-DC). The bound proteins were resolved using 8% SDS-PAGE and probed using Western blotting with the anti-FoxO1 monoclonal antibody.

### Data Analysis

In all biochemical studies, quadruplicate samples in each experimental group were assayed in three separate experiments. Means, standard errors, and standard devia tions were calculated for each experimental group. Statistical analysis was performed using one-way or two-way analysis of variance followed by Bonferroni's test or the Student t-test as appropriate (when only two sets of data were compared). P<0.05 was considered significant.

### Example 1

### Insulin Stimulates Deposition of Elastic Fibers in Cultures of Human AoSMCs

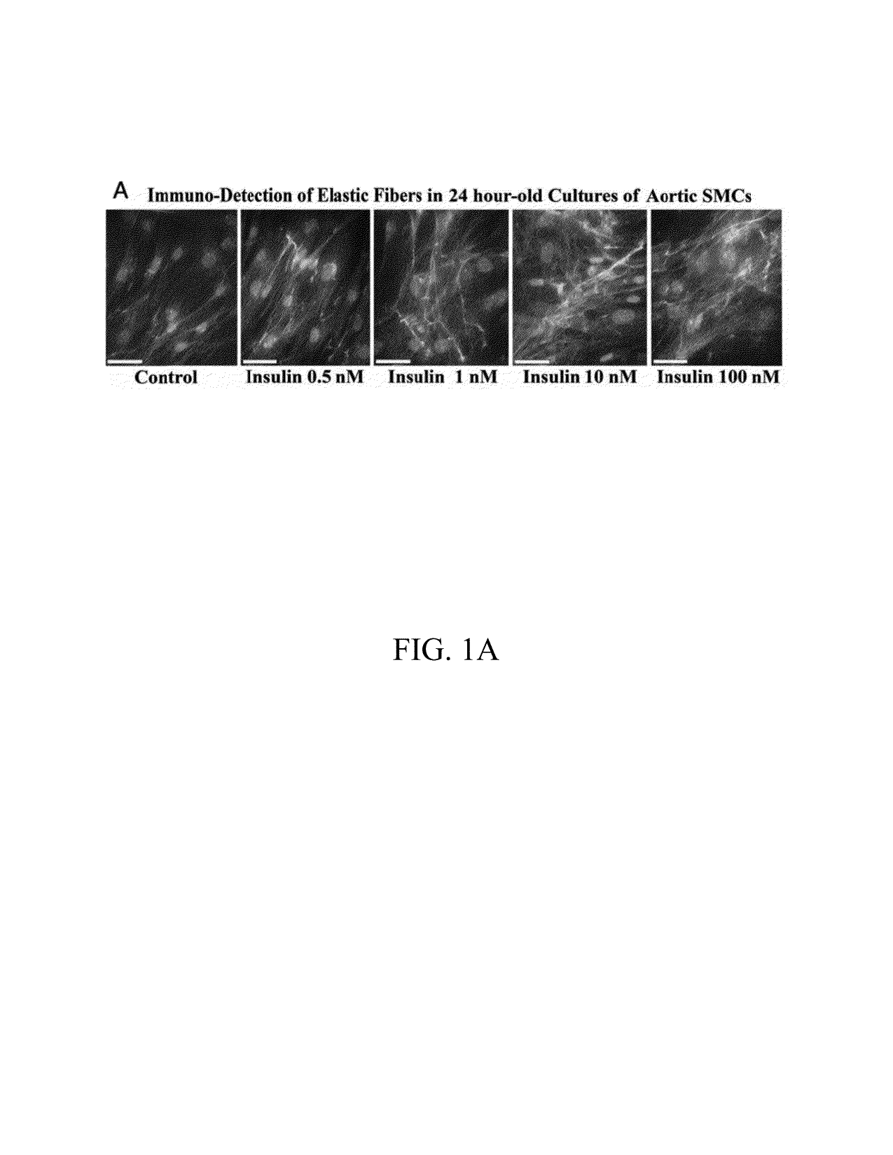

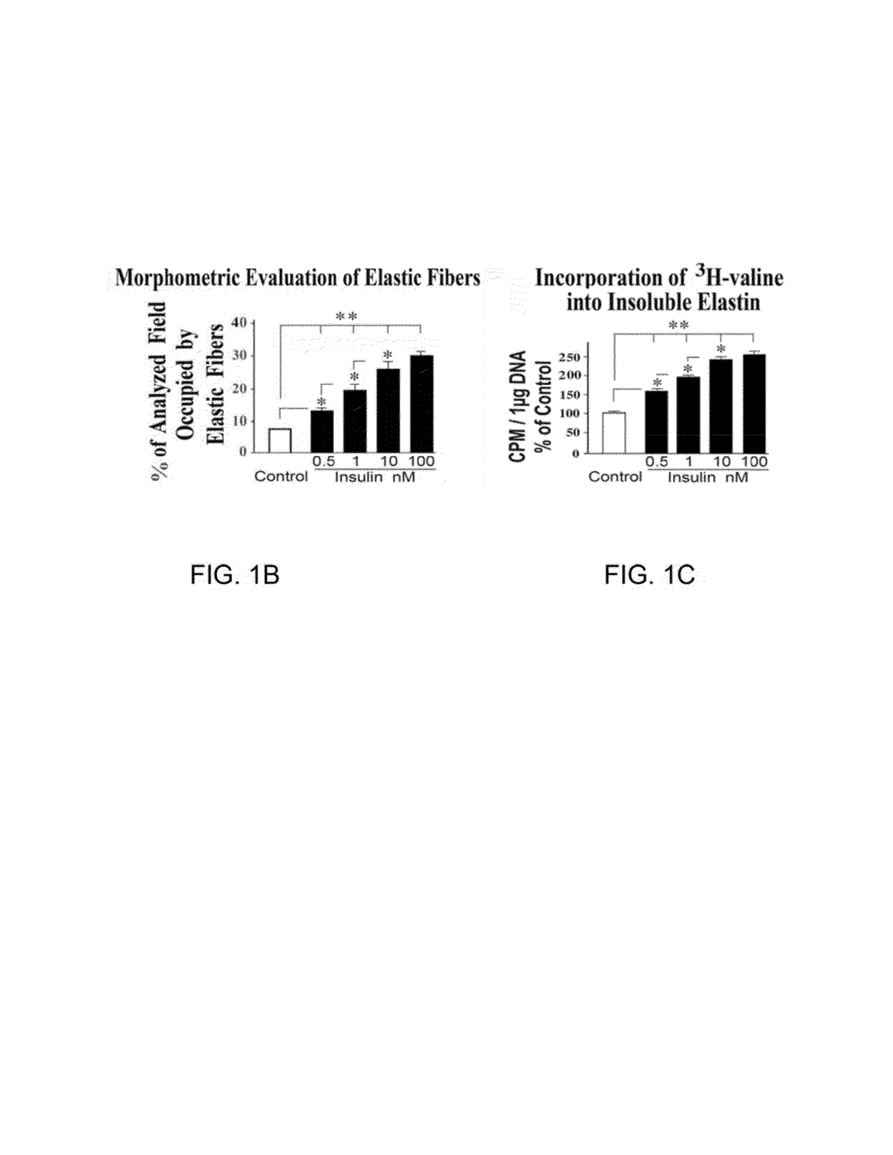

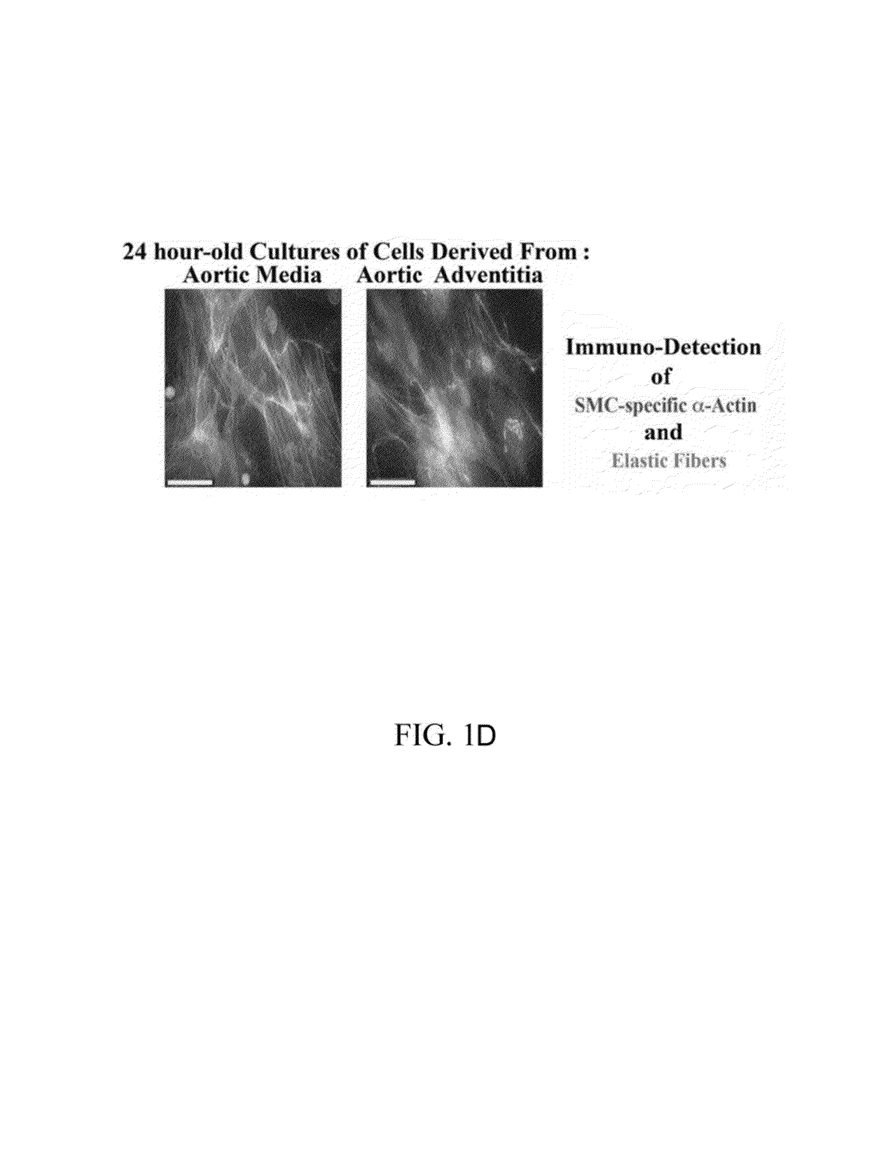

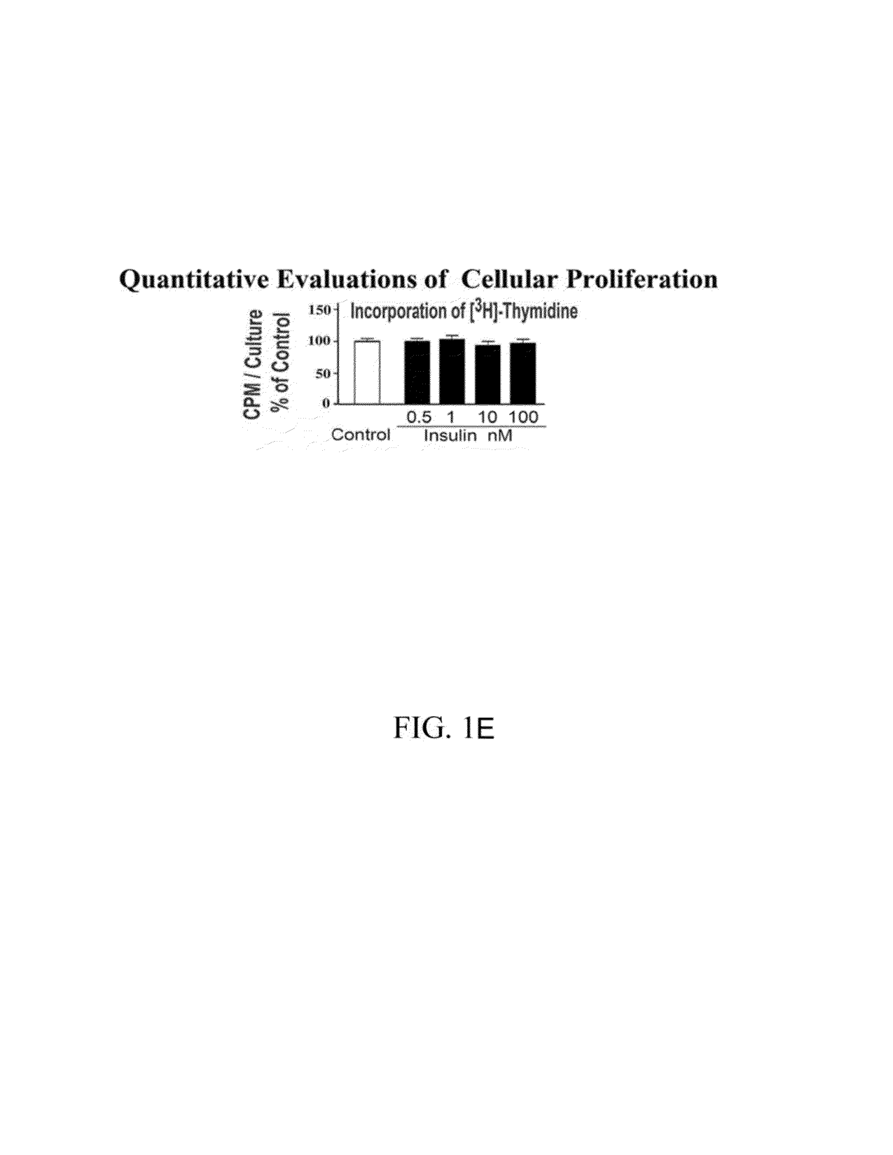

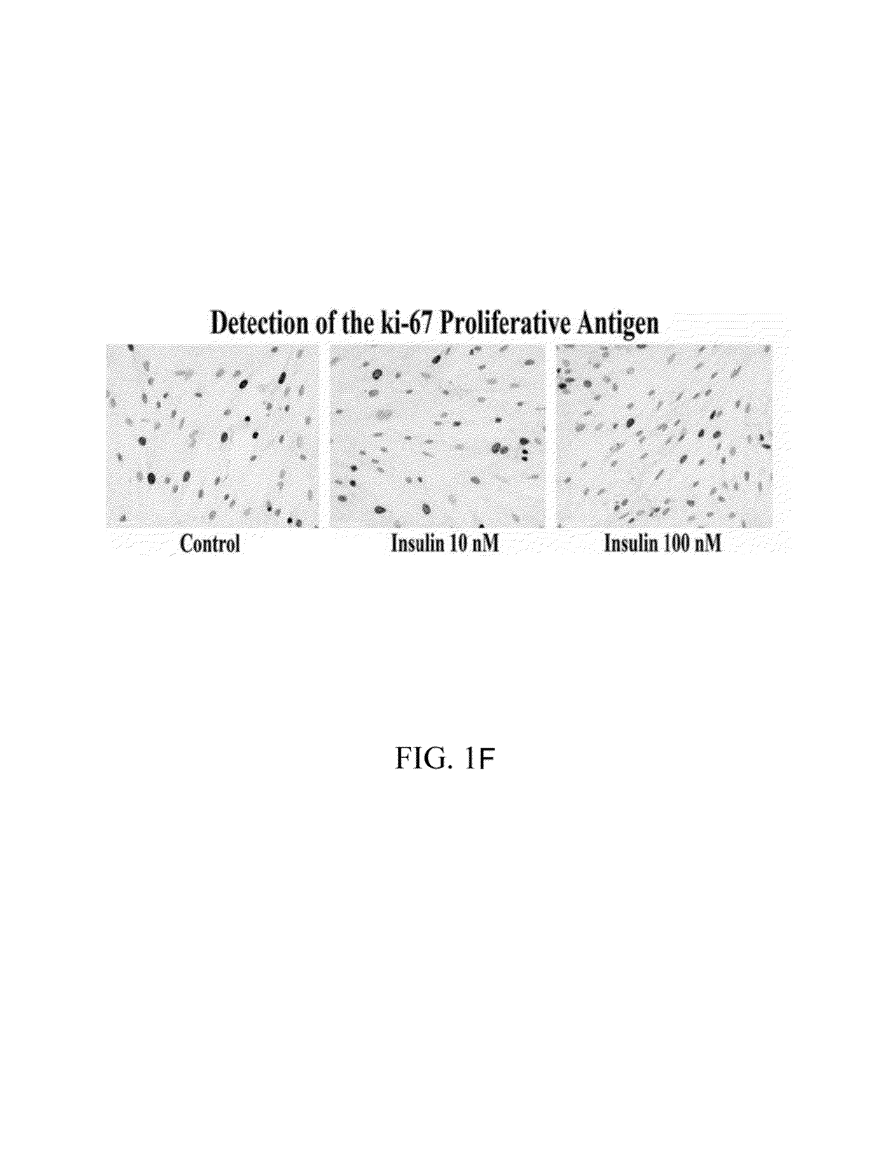

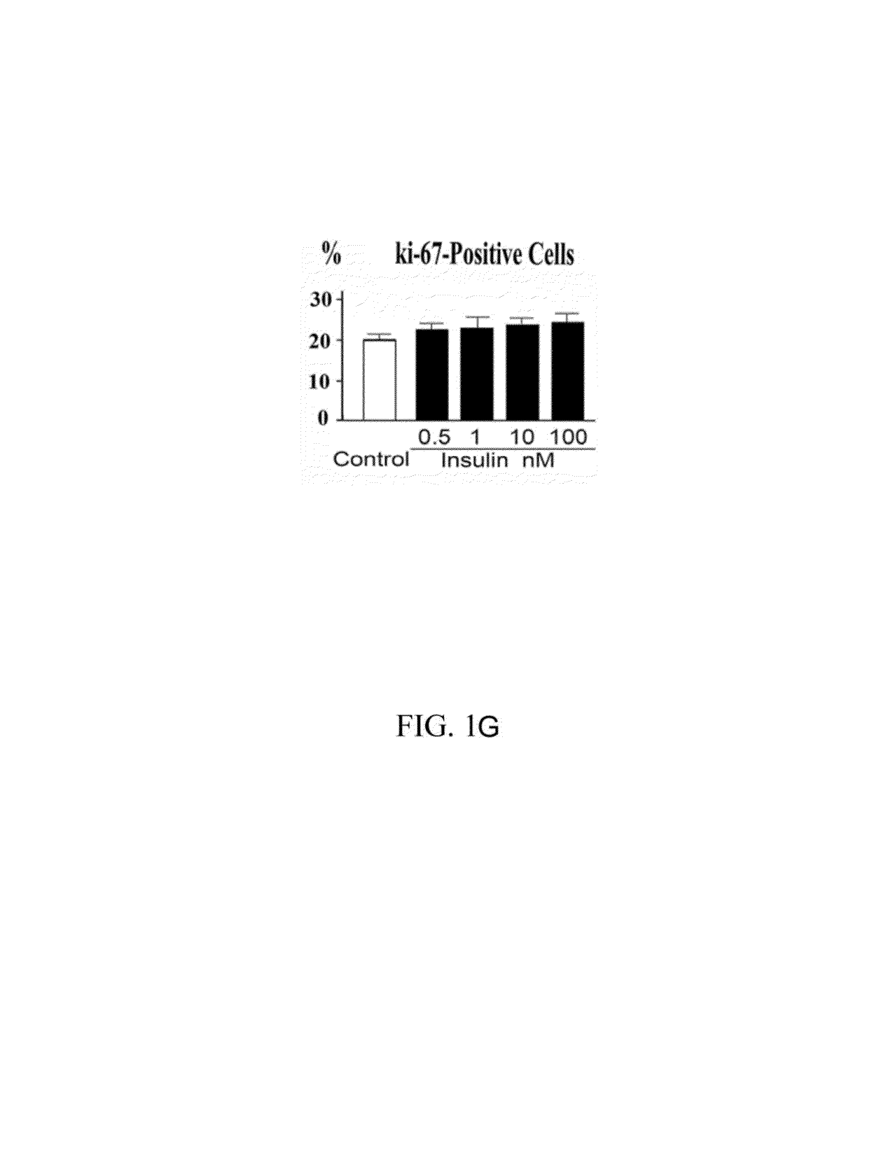

Results of the first series of experiments revealed that 24-hour treatment of confluent cultures of the aortic media-derived cells with 0.5 to 100 nmol/L insulin induced a dose-dependent increase in their production of the immunodetected elastic fibers and up-regulated levels of metabolically labeled insoluble elastin (FIG. 1A-C). We also documented that 95%±3% of cells present in the first, second, and third passages of cells derived from the original aortic media explants demonstrated abundant expression of the SMC-specific α-actin. However, results of additional tests revealed that in addition to a potent elastogenic effect on these aortic media-derived SMCs, 1 nmol/L insulin also stimulated deposition of new elastic fibers in the parallel cultures derived from aortic adventitia, which contained primarily fibroblasts and less than 10% of the α-actin-positive SMCs (FIG. 1D-E). In contrast, the insulin-treated cultures of both cell types did not reveal any up-regulation in deposition of immunodetectable fibronectin or collagen I. Results of the quantitative assay of [3H]-thymidine incorporation and immunodetection of Ki-67 proliferative antigen in 48-hour cultures of AoSMCs demonstrated that treatment with 0.5 to 100 nmol/L insulin did not induce any up-regulation in their basic proliferative rate (FIG. 1F-G).

### Example 2

### Insulin Stimulates Elastogenesis Through Activation of Insulin Receptor and Triggers a Downstream Signaling Pathway that Includes PI3K

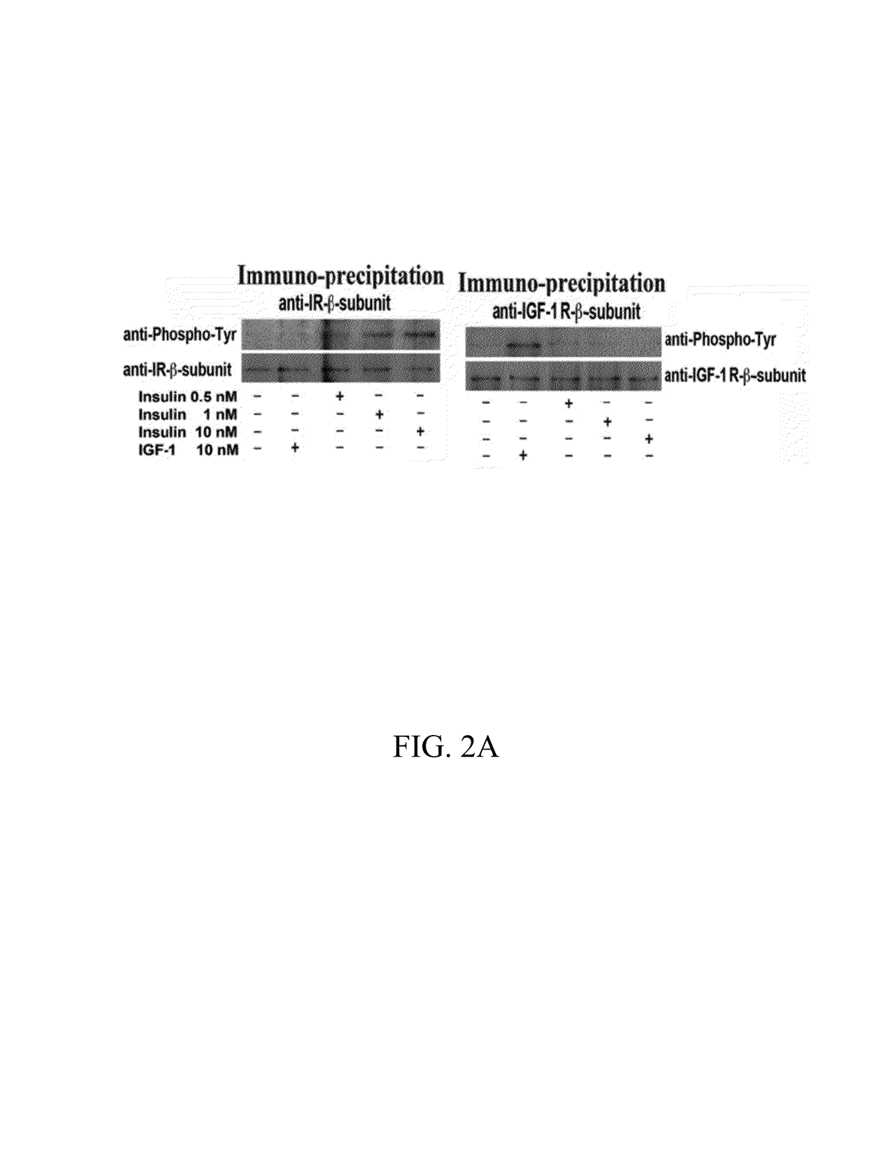

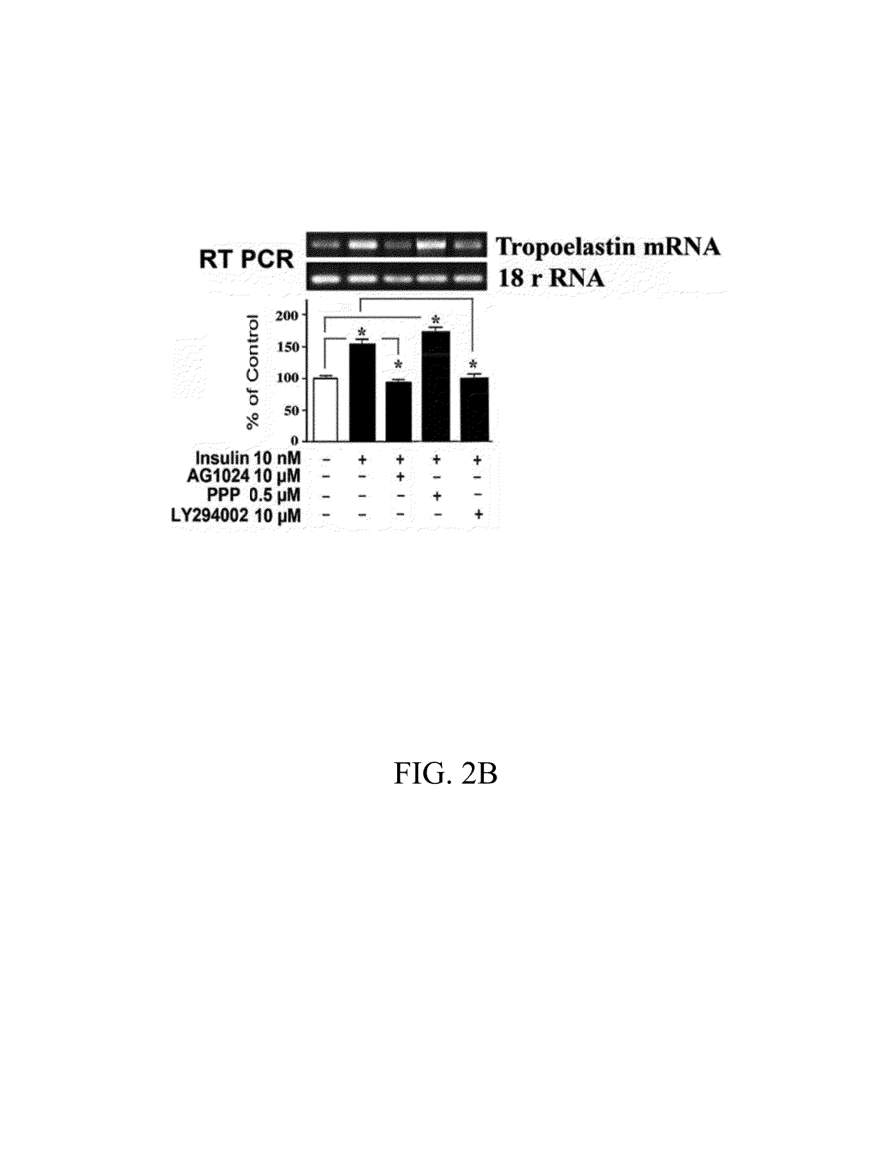

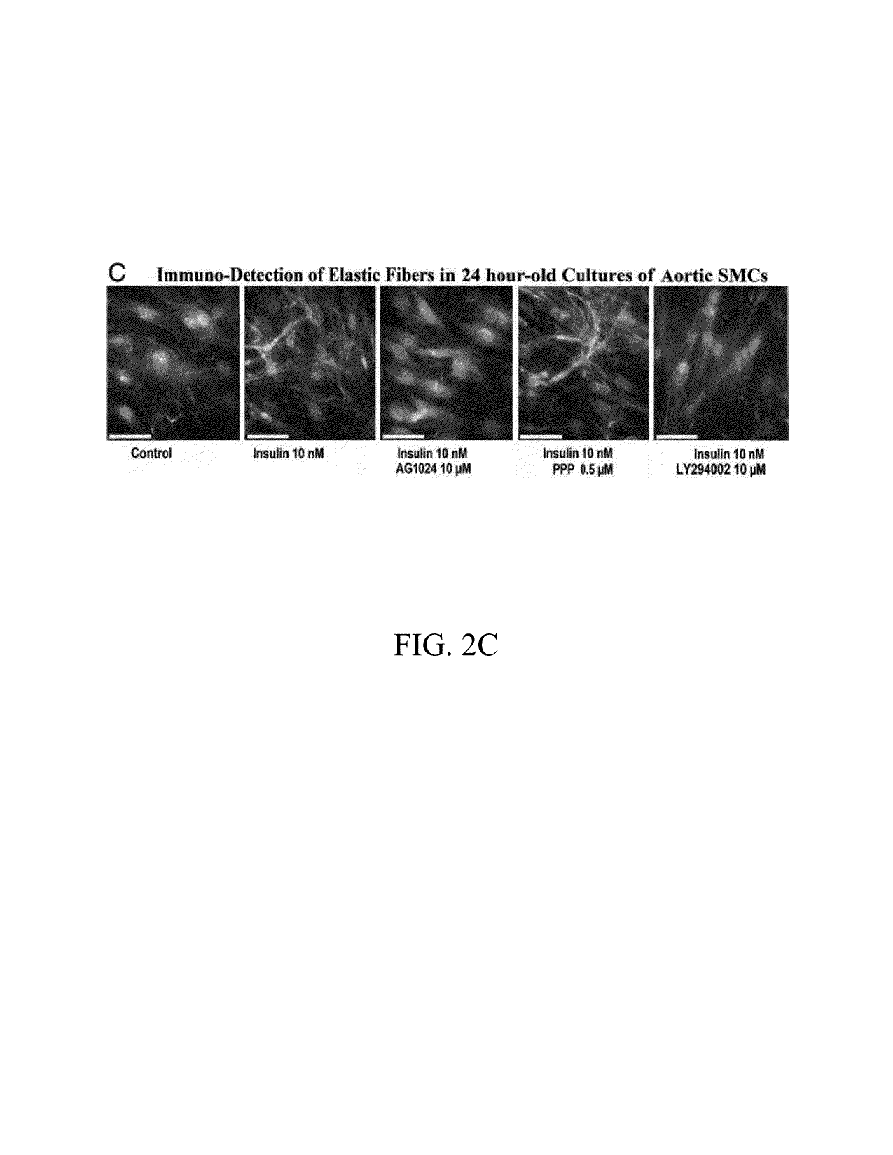

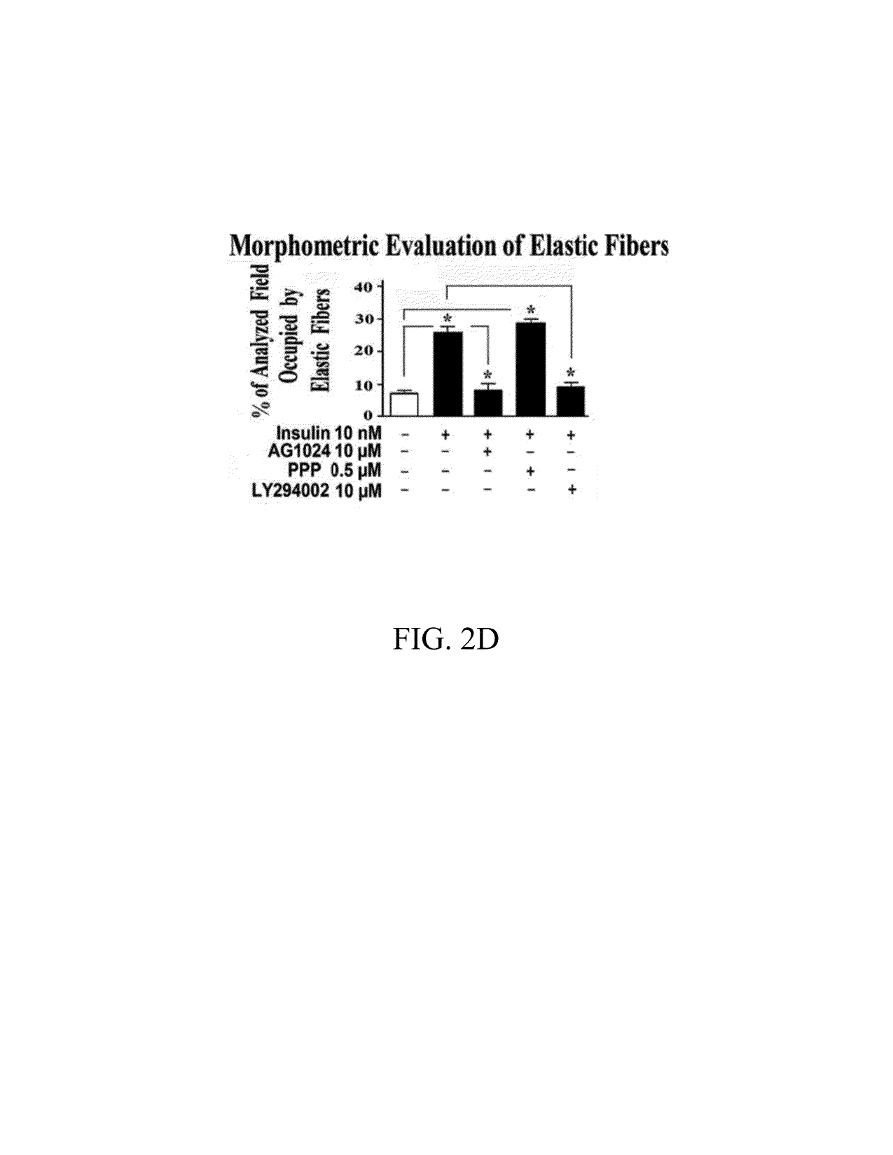

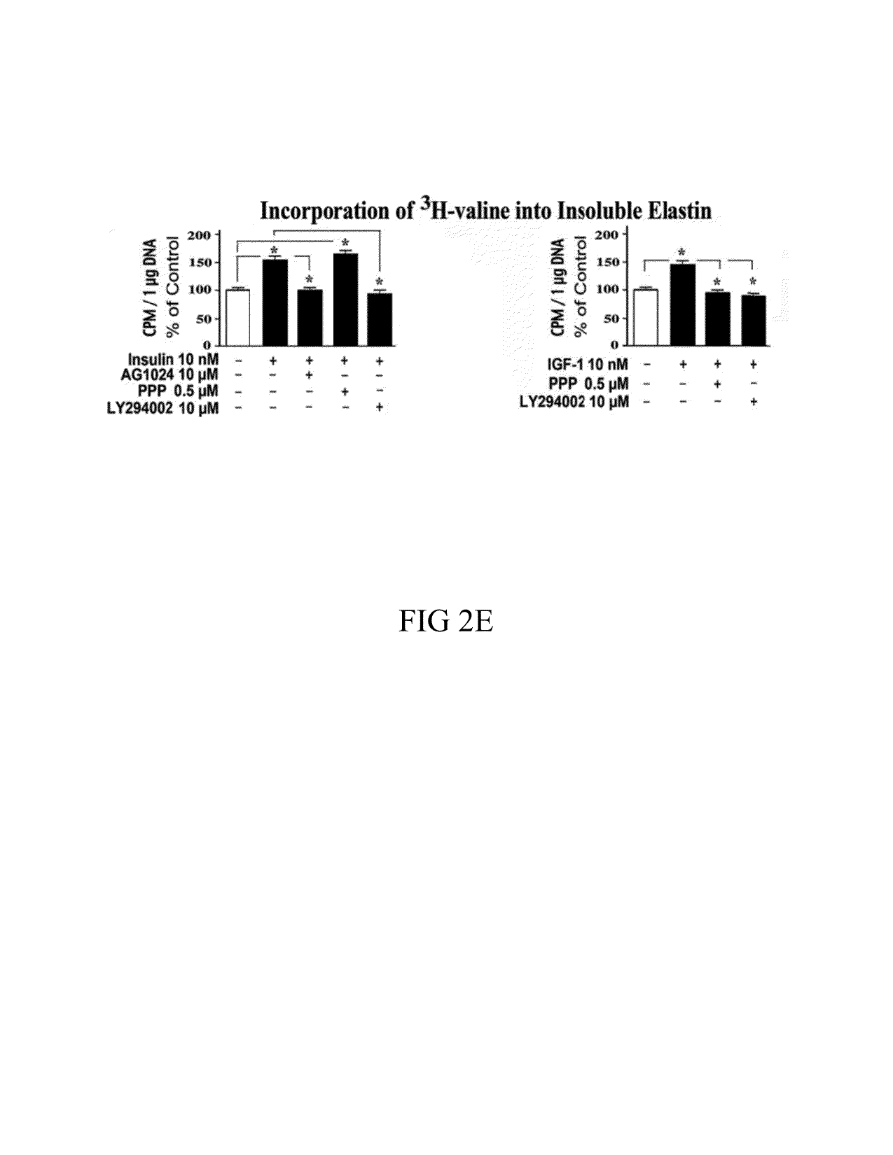

Because IGF-1 is a potent stimulator of elastogenesis and both insulin and IGF-1 can cross-activate their highly homologous receptors when applied in micromolar concentrations. Also tested was whether the observed up-regulation in net deposition of elastin would also be due to the cross-activation of IGF-1R by insulin. Results of experiments in which parallel immunoprecipitation using antibodies to beta subunits of insulin receptors and IGF-1R were followed by Western blotting using anti-phospho-Tyr antibody demonstrated that nanomolar concentrations of insulin induced dose-dependent phosphorylation of the insulin receptor, observed 30 minutes after addition of insulin, but did not affect phosphorylation of IGF-1R, which was phosphorylated only in cultures treated with IGF-1 (FIG. 2A).

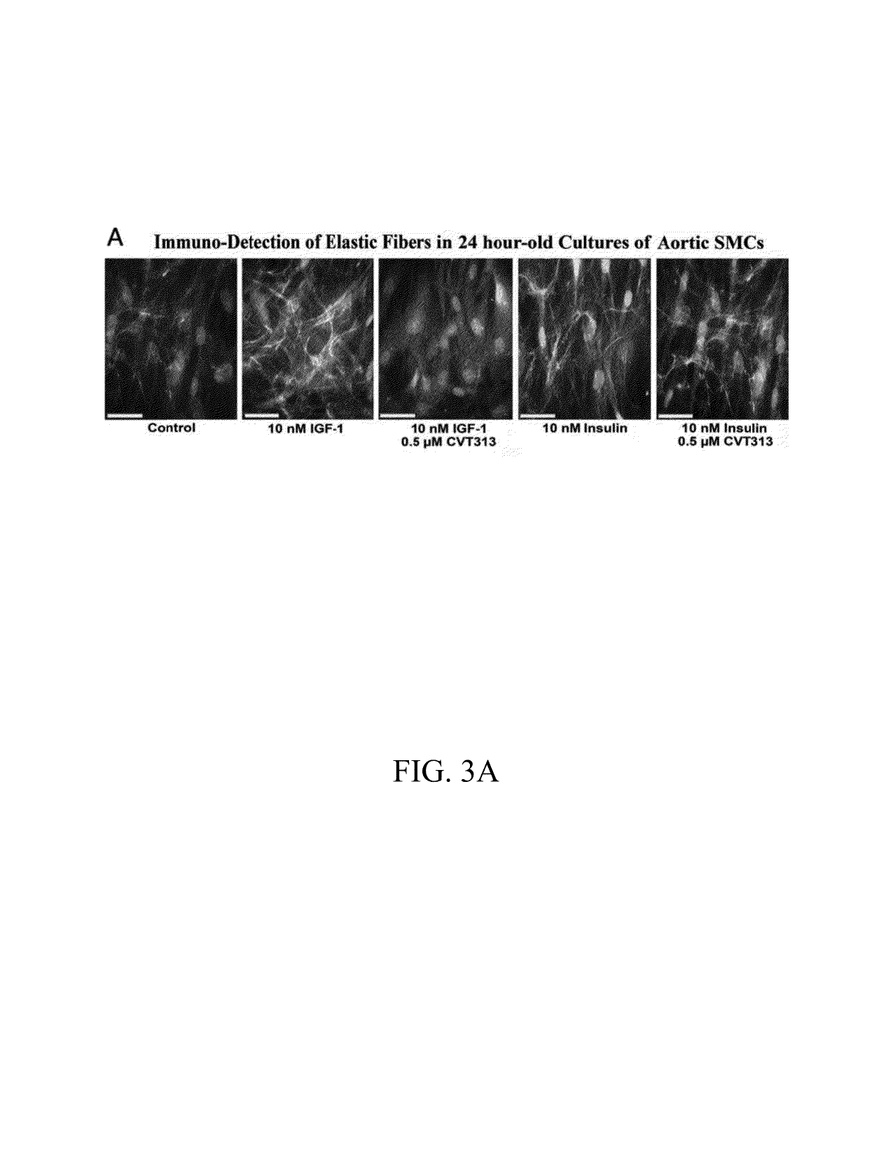

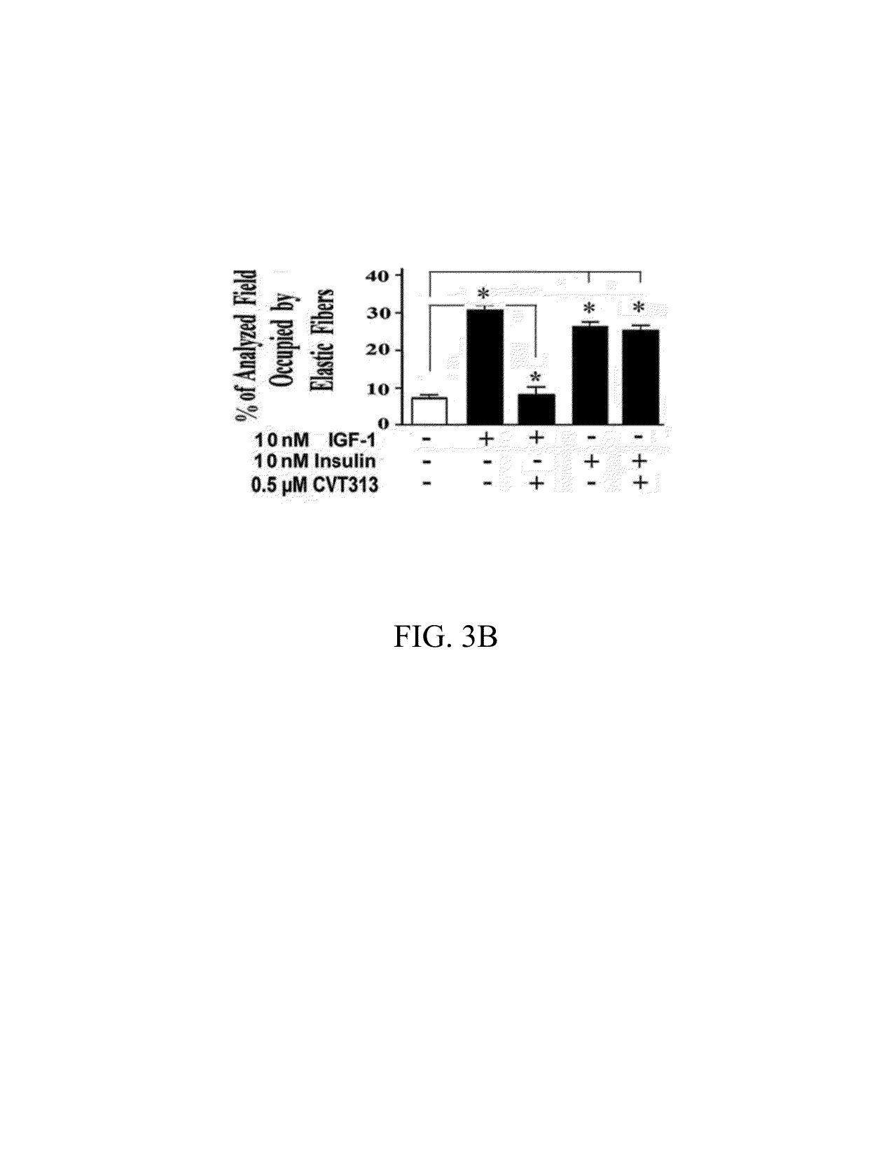

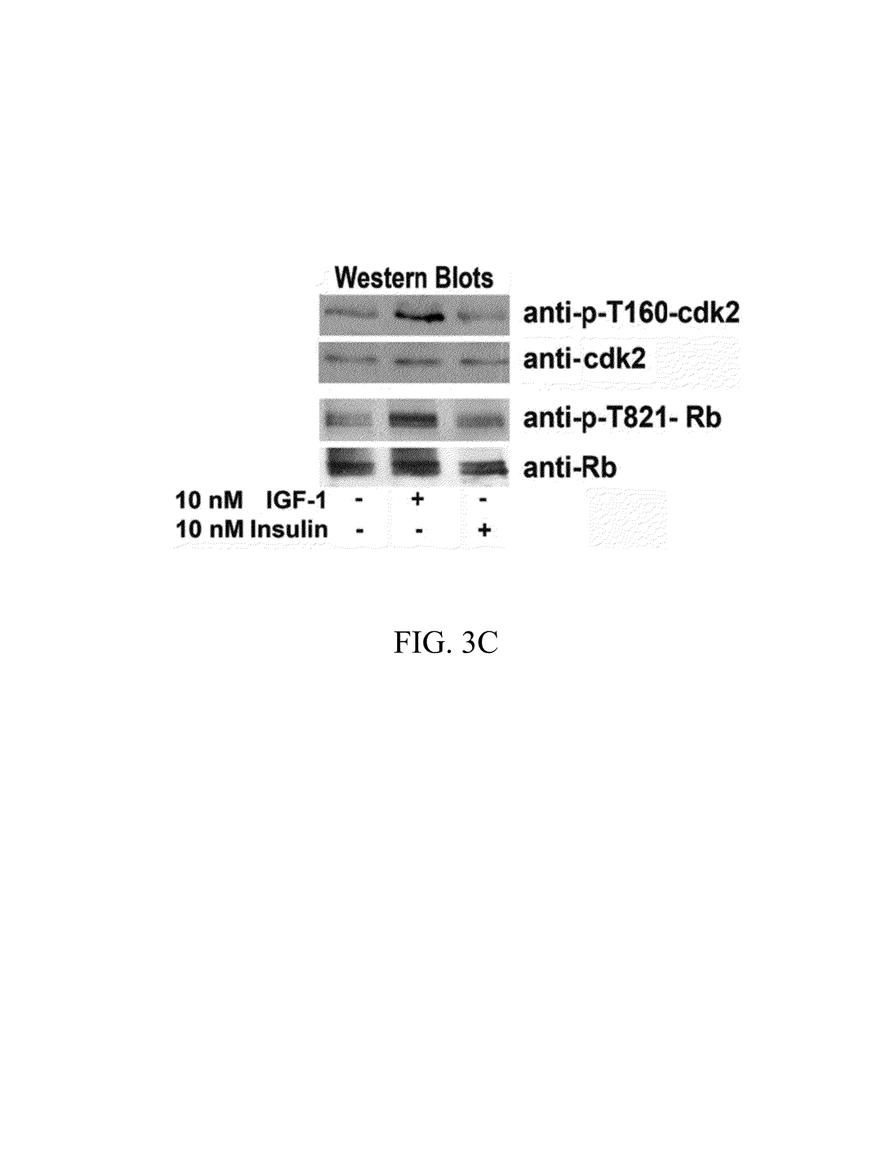

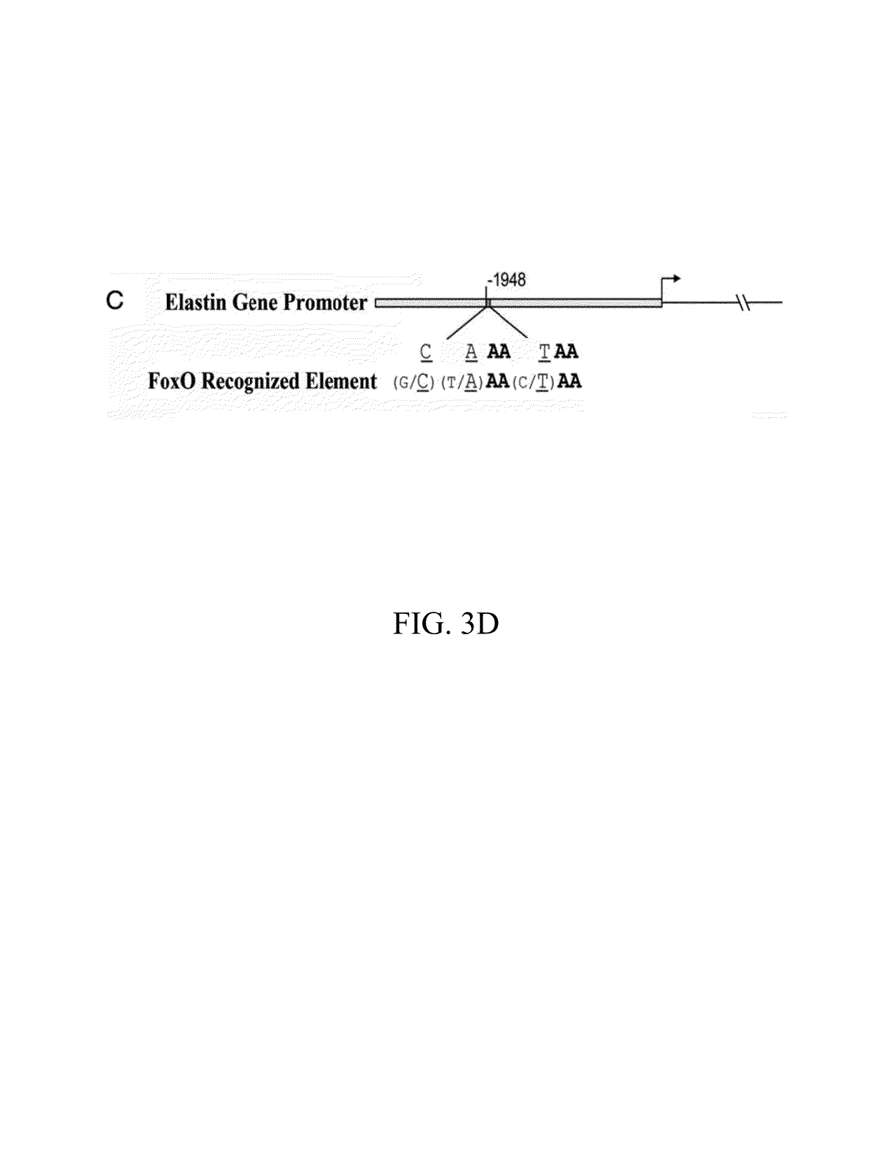

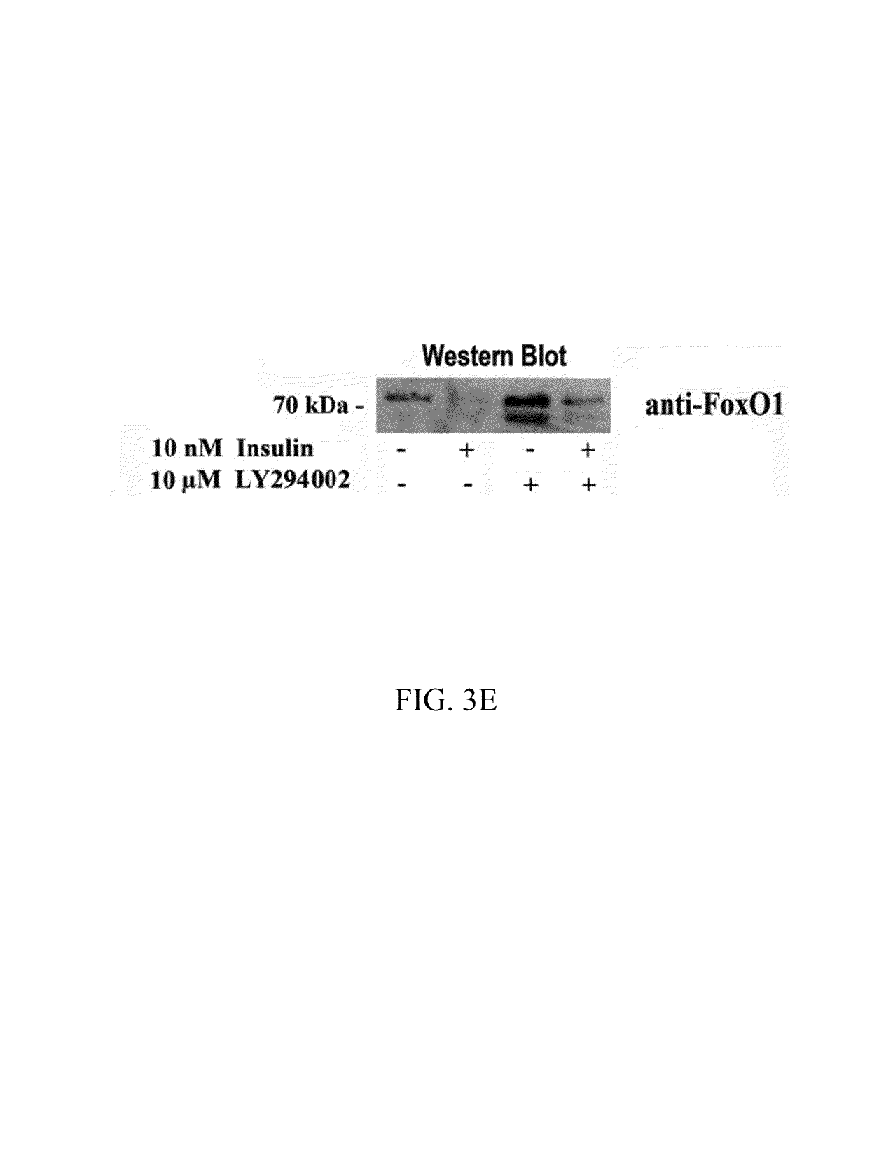

Results of quantitative RT-PCR assays, monitoring of the elastogenic response of AoSMCs to insulin in the time course, revealed that 10 nmol/L insulin caused significant up-regulation of the tropoelastin mRNA level in 4 hours, and the peak of this effect was observed in cultures treated for 8 hours. Inasmuch as pretreatment of parallel cultures with transcription inhibitor (50 μmol/L dichloro-benzimidazole riboside) completely abolished the insulin-induced up-regulation of tropoelastin mRNA levels, the result was that insulin causes initiation of elastin gene transcription and does not promote tropoelastin mRNA stability. Then, it was shown that elastogenic effects of 10 nmol/L insulin, observed at the mRNA level, were abrogated in cultures pretreated and subsequently maintained using AG1024, which inhibits phosphorylation of the insulin receptor, or with a highly selective inhibitor of PI3K (LY294002). In contrast, pre-treatment and incubation of parallel cultures with the specific inhibitor of IGF-1R tyrosine kinase (picropodophyllin) did not inhibit the elastogenic effects induced by 10 nmol/L insulin (FIG. 2B). Results of quantitative immunocytochemistry using anti-tropoelastin antibody (FIG. 2C) and assessment of [3H]-valine-labeled insoluble elastin further demonstrated that 10 nmol/L insulin induces the ultimate deposition of elastin through activation of its own receptors and triggers a signal that involves PI3K (FIG. 2D). In contrast, it was also demonstrated that inhibition of PI3K (with LY294002) also abolished the elastogenic effects induced by IGF-1 (FIG. 2E). Thus to an extent, both elastogenic signaling pathways, induced by either insulin or IGF-1, require activation of PI3K. However, results of the following series of experiments also indicated that the consecutive steps of these two pathways, downstream of PI3K, may diverge and ultimately initiate the elastin gene transcription in different ways. It has been suggested that the final steps of the IGF-1-induced elastogenic signaling pathway leads to activation of a regulatory element on tropoelastin gene promoter (retinoblastoma control element). In addition, the IGF-1-initiated elastogenic signal includes activation of the cyclin E/cdk-2 complex that causes site-specific phosphorylation of retinoblastoma on threonine 821, a prerequisite step that consecutively enables the binding and delivery of the Sp1 transcription activation factor to the retinoblastoma control element and initiation of elastin gene transcription. Herein it is demonstrated that in contrast to IGF-1-treated AoSMCs, in which specific inhibition of cdk-2 with CVT313 abolished the final elastogenic effect of this growth factor, the insulin-treated cells still exhibited heightened deposition of elastic fibers when cultured in the presence of the same cdk-2 inhibitor (FIG. 3A-B). In addition, it was established that, in contrast to IGF-1, insulin did not induce phosphorylation of cdk-2 on tyrosine 160, required for activation of this kinase, or promote the site-specific phosphorylation of retinoblastoma on threonine (FIG. 3C). As such, insulin would induce activation of the elastin gene through the action of a different control element located within the elastin gene promoter than the IGF-1.

### Example 3

### Insulin Induces Dissociation of FoxO1 Transcription Inhibitor from the FoxO-Recognized Element, Identified within the Elastin Gene Promoter

Insulin initiates transcription of numerous genes via the mechanism causing detachment of the transcription inhibitors (belonging to the forkhead box O (FoxO) superfamily) from their unique domains, FoxO-recognized elements (FREs) located in the cis-regulatory regions of their promoters. We identified that the CAAATAA sequence located on position-1948 in the human elastin gene promoter is highly homologous to the FRE consensus (G/C)(T/A)AA(C/T)AA described in numerous insulin-responsive genes (FIG. 3D). Most importantly, it was observed that the synthetic DNA probe reflecting the putative FRE detected in elastin gene promoter (immobilized on Dynabeads) bound and sequestered the protein (interacting with anti-FoxO1 antibody) from the nuclear extracts of AoSMCs incubated in the absence of insulin. Meaningfully, the beads-immobilized DNA probe could not bind FoxO1 from nuclear extracts of cells treated for 30 minutes with 10 nmol/L insulin. In contrast, this probe bound abundant FoxO1 proteins from the nuclear extracts of cells preincubated using the PI3K inhibitor LY294002. The consecutive incubation of LY294002-treated cells with insulin could not reverse this interaction. These data (FIG. 3E) show that insulin induces PI3K-dependent phosphorylation of FoxO1 protein, thereby preventing its binding to the elastin gene promoter-derived DNA probe with FRE consensus.

### Example 4

### Insulin-Induced and PI3K-Dependent Signals Also Up-Regulate Secretion of Tropoelastin

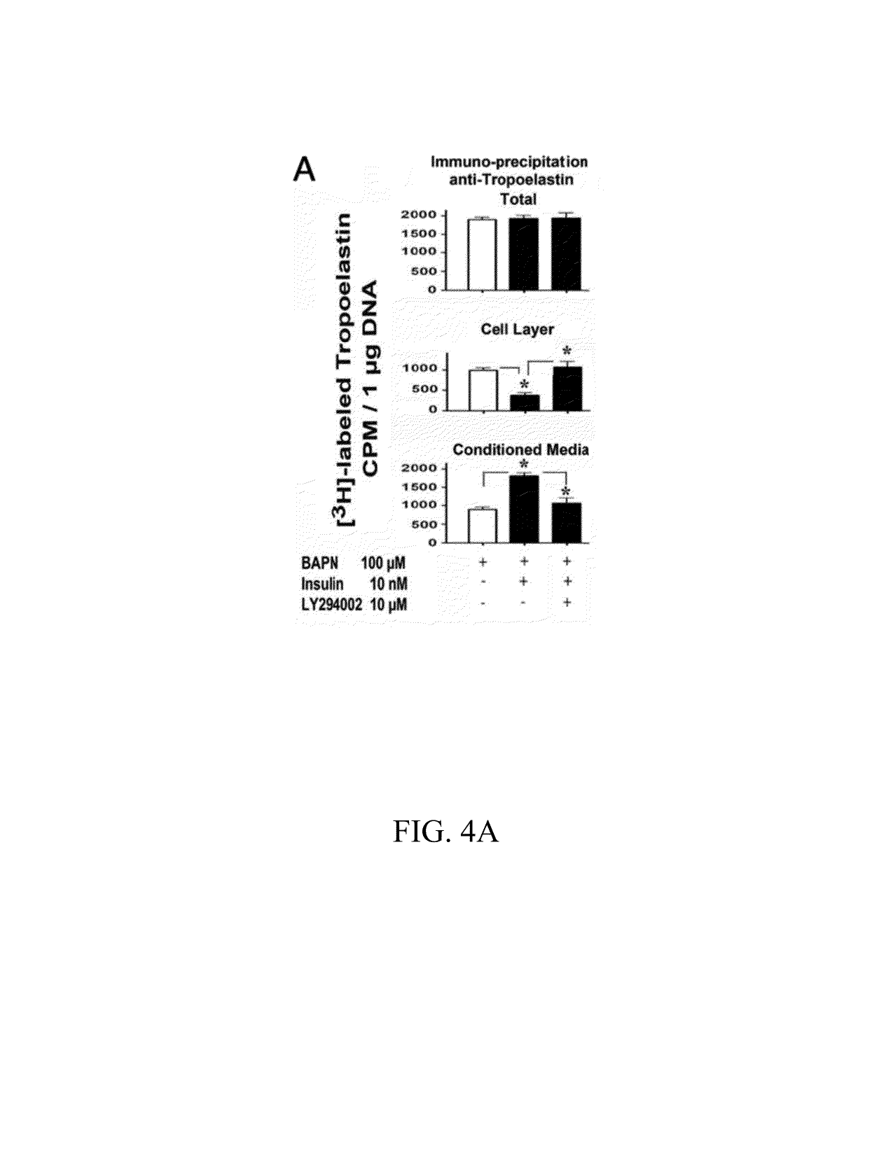

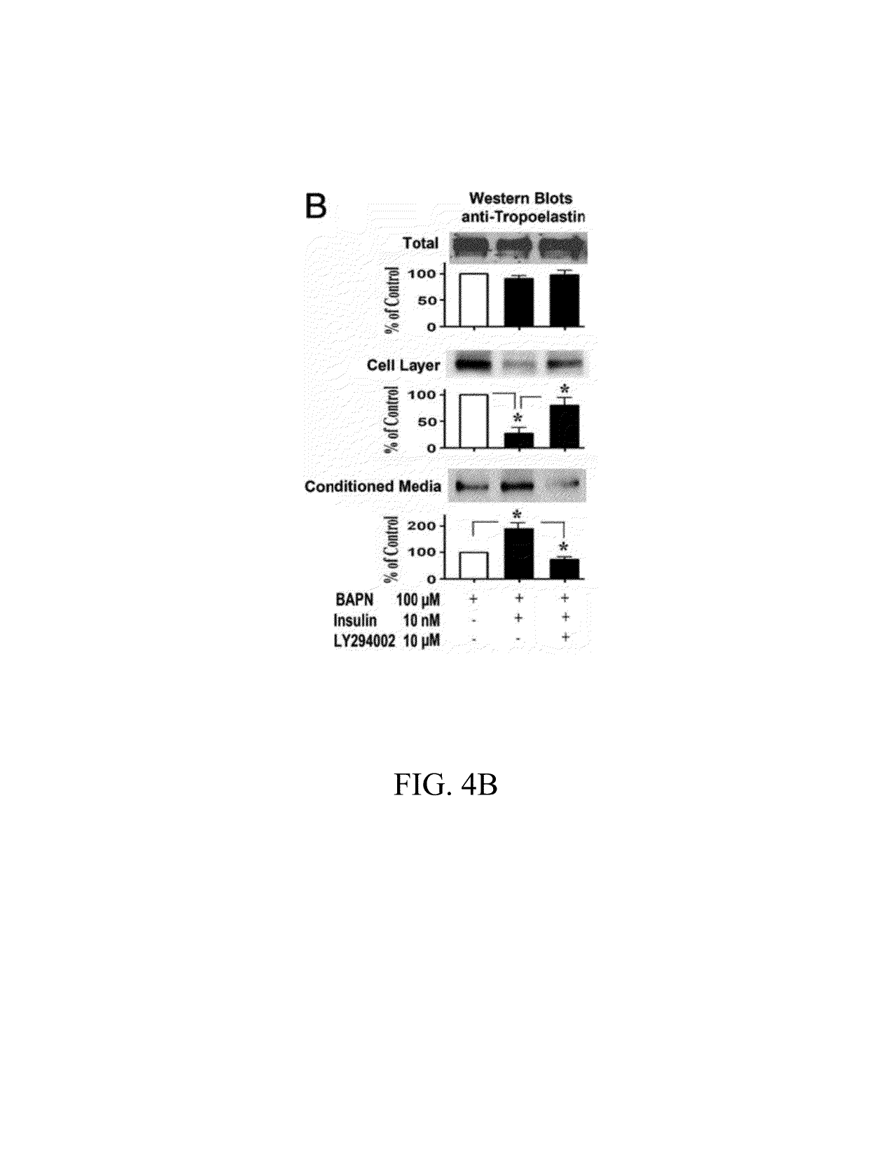

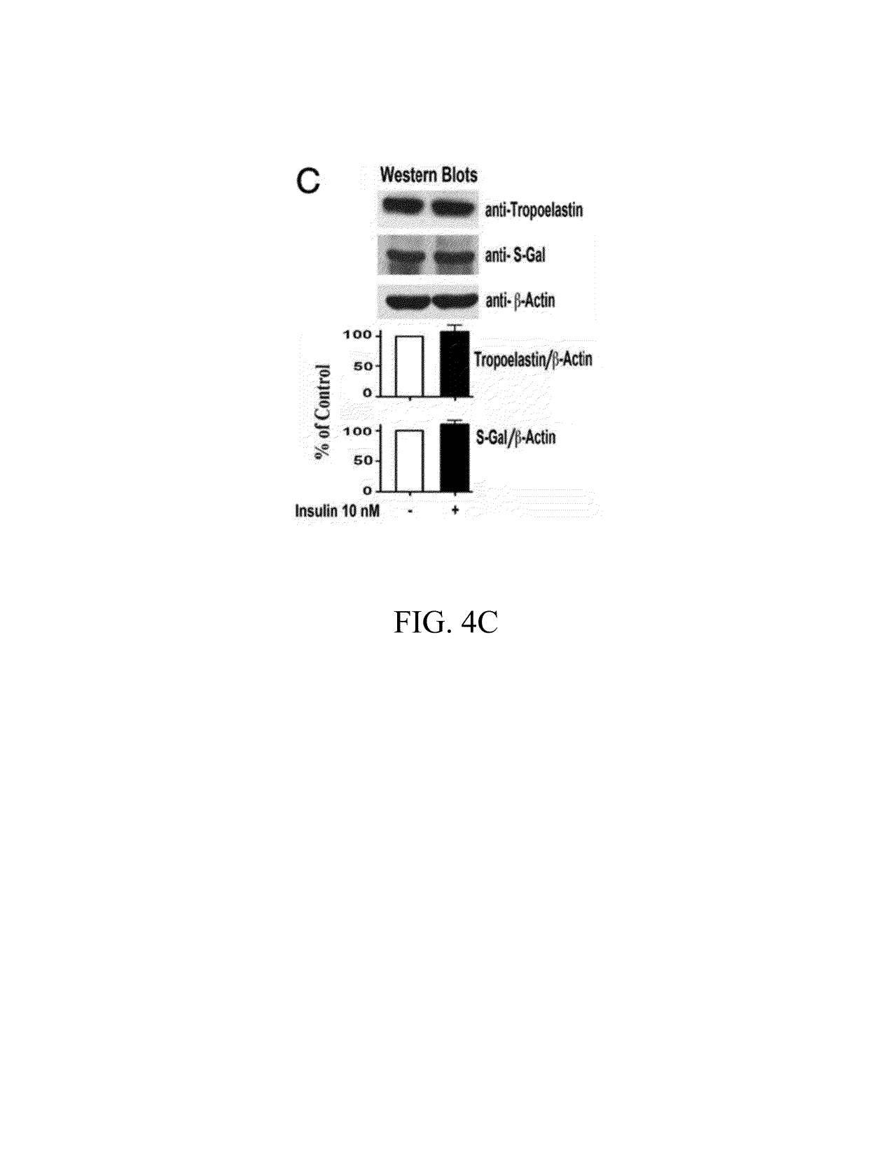

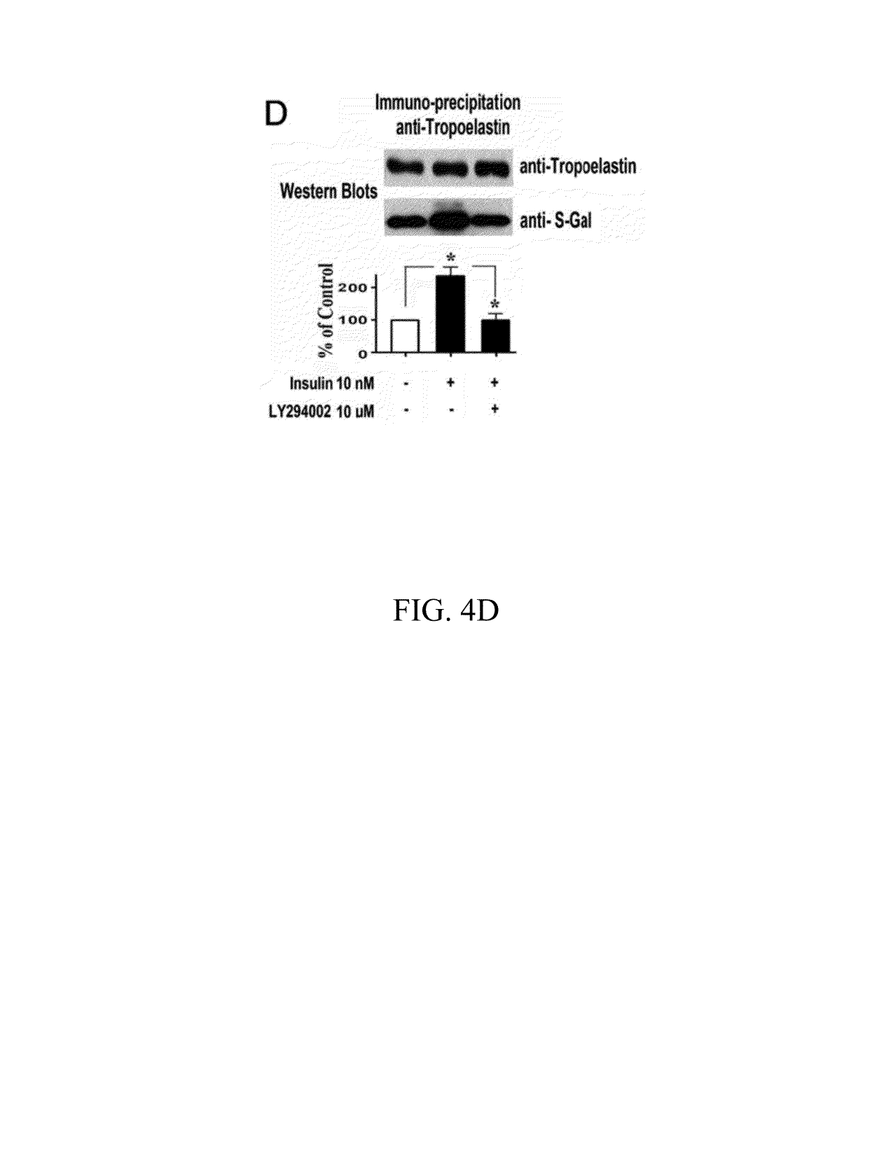

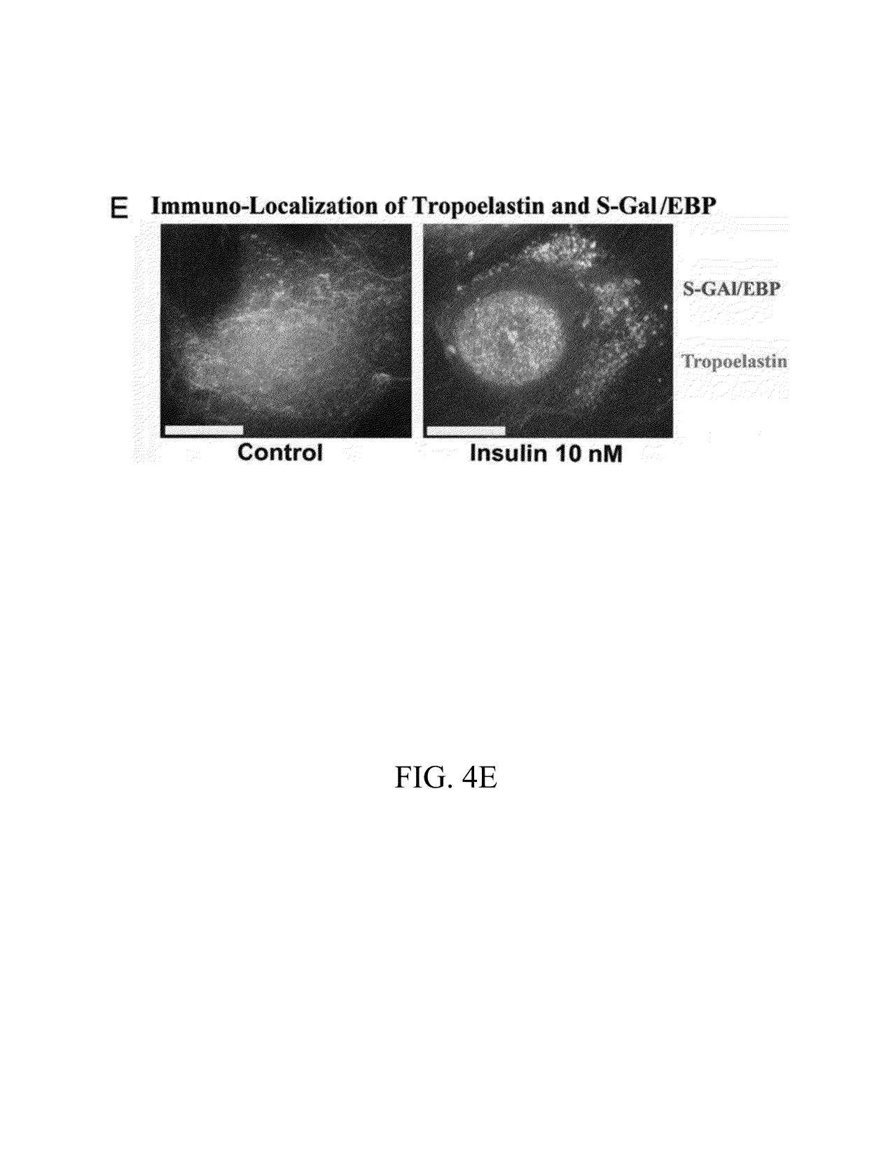

In the next series of experiments, it was determined whether insulin-triggered signals would also affect tropoelastin secretion. Confluent cultures of AoSMCs maintained in medium with 2% FBS were first incubated for 3 hours with [3H]-valine to metabolically label their newly synthesized tropoelastin and with an inhibitor of lysyl oxidase, β-aminoproprionitrile, to prevent its cross-linking. Then, parallel cultures were incubated in the presence and absence of 10 nmol/L insulin for 1 hour. We observed that the cell layer extracts of these insulin-treated AoSMCs contained (on average, 66%±6%) less metabolically labeled tropoelastin than did the untreated controls. However, their conditioned media, assessed at the same time, contained significantly (94%±4%) more metabolically labeled tropoelastin than did their untreated counterparts (FIG. 4A). The proportional amounts of immunoprecipitated tropoelastin detected in each fraction were additionally confirmed via quantification of the Western blots (FIG. 4B). These insulin-induced changes in levels of the already synthesized tropoelastin could not be observed in parallel cultures, in which PI3K activity was inhibited by 1-hour preincubation with LY294002. Together, the present data indicate that the insulin-induced signaling cascade that involves activation of PI3K also triggers cellular mechanisms that facilitate secretion of the already synthesized tropoelastin.

### Example 5

### Insulin-Induced and PI3K-Dependent Signals Promote Intracellular Association Between Tropoelastin and its S-Gal/EBP Chaperone

Hydrophobic and unglycosylated tropoelastin must be chaperoned through the secretory pathways by the 67-kDa EBP, identified as the catalytically inactive spliced variant of S-Gal. Therefore, it was tested whether insulin could also modulate interactions between tropoelastin and its EBP/S-Gal chaperone. Results of experiments in which confluent cultures of AoSMCs were incubated for only 20 minutes in the presence or absence of 10 nmol/L insulin demonstrated that the cell extract of insulin-treated cultures did not demonstrate any increase in basic levels of intracellular S-Gal/EBP or tropoelastin (assessed using Western blot analysis) (FIG. 4C). However, at the same time, significantly more S-Gal/EBP could be co-immunoprecipitated using anti-tropoelastin antibody from the ex-tracts of insulin-treated cells than from untreated cultures. This effect of insulin could not be induced in parallel cultures of cells pretreated for 30 minutes with PI3K inhibitor (FIG. 4D). Further analysis of parallel cultures of with S-Gal/EBP in numerous peripheral vesicles (yellow fluorescence). Inasmuch as the insulin treatment could not induce such tropoelastin/S-Gal/EBP co-localization in cells preincubated using LY294002 (data not shown), the data show that insulin-activated pathways that involve PI3K also facilitate transportation of tropoelastin into the secretory endosomes, where it can meet the S-Gal/EBP chaperone.

Although the present invention has been described in considerable detail with reference to certain preferred embodiments thereof, other versions are possible. Therefore, the spirit and scope of the appended claims should not be limited to the description and the preferred embodiments disclosed herein.

What is claimed is:

1. A method of stimulating cellular elastogenesis comprising delivering an insulin receptor agonist to a cell having an insulin receptor thereby inducing elastogenesis.

2. The method of claim 1, wherein the insulin receptor agonist is insulin.

3. The method of claim 2, wherein the insulin is delivered at a concentration of 0.5 nM-10 nM.

4. The method of claim 1, wherein the insulin agonist is selected from the group consisting of an insulin analogue, an insulin fragment, an insulin alpha chain, an insulin beta chain, pro-insulin, pre-pro-insulin, porcine insulin, bovine insulin, human insulin, synthetic insulin and combinations thereof.

5. The method of claim 1, wherein the insulin receptor agonist is delivered ex vivo.

6. The method of claim 1, wherein the insulin receptor agonist is delivered in vivo.

7. The method of claim 1, wherein the insulin receptor agonist is delivered to cells locally.

8. The method of claim 1, wherein the insulin receptor agonist is delivered to cells systemically.

9. The method of claim 7, wherein the concentration of insulin receptor agonist is delivered at 0.5 nM-10 nM.

10. A method of stimulating elastogenesis in a patient comprising delivering an insulin receptor agonist to cells of the patient.

11. The method of claim 10, wherein the cells are selected from the group consisting of smooth muscle cells, fibroblasts, and skin cells.

12. The method of claim 11, wherein the concentration of insulin receptor agonist is delivered at 0.5 nM-10 nM.

13. The method of claim 11, wherein the concentration of insulin receptor agonist does not stimulate the insulin-like growth factor 1 receptor.

14. The method of claim 10, wherein the insulin agonist is selected from the group consisting of an insulin analogue, an insulin fragment, an insulin alpha chain, an insulin beta chain, pro-insulin, pre-pro-insulin, porcine insulin, bovine insulin, human insulin, synthetic insulin and combinations thereof.

15. The method of claim 10 wherein the patient has an elastinopathy selected from the group consisting of atherosclerosis, ischemic neuropathy, ischemic heart disease, peripheral vascular disease, cerebrovascular disease, ulceration, chronic wounds, ischemic tissue, metabolic syndrome, diabetic-associated retinopathy, diabetic-associated arteriosclerosis, diabetic-associated radiculopathy, diabetic-associated neuropathy supravalvular aortic stenosis (SVAS), Williams-Beuren syndrome (WBS), Cutis Laxa, Marfan disease, GM-1-gangliosidosis, Morquio B, Hurler disease, Costello syndrome, Ehlers Danlos syndrome, and pseudoxanthoma elasticum (PXE).

16. A pharmaceutical composition comprising:
an insulin receptor agonist at a dosage delivering 0.5 nM-10 nM of agonist to tissue; and
a pharmaceutically acceptable excipient.

17. The pharmaceutical composition of claim 16, wherein the agonist increases the net deposition of elastin cells.

18. The pharmaceutical composition of claim 17, wherein the insulin agonist is selected from the group consisting of an insulin analogue, an insulin fragment, an insulin alpha chain, an insulin beta chain, pro-insulin, pre-pro-insulin, porcine insulin, bovine insulin, human insulin, synthetic insulin and combinations thereof.

19. The pharmaceutical composition of claim 16, wherein the pharmaceutical composition is formulated to be administered by a mode selected from the group consisting of topical, parenteral, subcutaneous, intravenous, intraperitoneal, transdermal, oral, buccal, inhalation, depot injection, and implantation.

20. The pharmaceutical composition of claim 17, wherein the concentration of insulin receptor agonist does not stimulate the insulin-like growth factor 1 receptor in the patient.
